(19) United States
(12) Patent Application Publication
(10) Pub. No.: US 2021/0177803 A1
(43) Pub. Date: Jun. 17, 2021

(54) NEW PHARMACEUTICAL USE FOR THE TREATMENT OF HEART FAILURE

(71) Applicant: Novartis AG, Basel (CH)

(72) Inventors: Dmytro BUTYLIN, Basel (CH); Philippe FERBER, Willer (FR); Adele NOE, Huningue (FR)

(21) Appl. No.: 17/269,310

(22) PCT Filed: Aug. 22, 2019
(86) PCT No.: PCT/IB2019/057081

§ 371 Date: Feb. 18, 2021

(30) Foreign Application Priority Data
Aug. 23, 2018 (EP) 18190588.6
Oct. 10, 2018 (EP) 18199733.9

(51) Int. Cl. A61K 31/41; A61P 9/04; A61K 31/216

(52) U.S. Cl. CPC A61K 31/41; A61K 31/216; A61P 9/04

(57) ABSTRACT

The present invention relates to novel methods for the treatment of heart failure in a patient.

The present invention relates to novel methods and novel uses in the treatment of heart failure in a patient, in particular in a patient with heart failure (HF) with a reduced ejection fraction (EF) (HFrEF), wherein treatment with sacubitril and valsartan is initiated shortly after an acute decompensation heart failure episode of said patient.

### BACKGROUND OF THE INVENTION

Heart failure (HF) is a global pandemic with an estimated worldwide prevalence of 38 million patients (Ambrosy A P, et al. J Am Coll Cardiol 2014; 63:1123-33; Writing Group M, Mozaffarian D, et al. Circulation 2016; 133:e38-360). In the United States alone, there are more than 1 million admissions for HF as a primary diagnosis per year, representing 1%-2% of all hospitalizations (Blecker S, et al. J Am Coll Cardiol 2013; 61:1259-67; Gheorghiade M, et al. JAMA 2006; 296:2217-26). Despite available therapy, mortality and readmission rates within 60-90 days of discharge for patients hospitalized with heart failure (HF) approach 15% and 30%, respectively (Greene S J, et al. Nat Rev Cardiol 2015; 12:220-29).

Despite numerous promising clinical development programs, there have been relatively few major breakthroughs in the management of HF in the acute setting, and the cornerstone of therapy remains intravenous (IV) diuretics, vasodilators, and less commonly inotropes (Vaduganathan M, et al. Nat Rev Cardiol 2013; 10:85-97). A hospitalization for decompensation of HF increases the risk of cardiovascular (CV) death by almost 3 times, and the risk is especially high during the first 30 days after discharge This early postdischarge period has been termed the ‘vulnerable phase’ and accounts for a disproportionate amount of the >US$30 billion spent annually on HF care in the USA. (Ahmed, et al. J Card Fail 2008; 14:211-18; Greene et al. Nat Rev Cardiol 2015; 12:220-29). To address this vulnerable phase, the ESC guidelines recommend the optimization of chronic HF treatment while the patient is hospitalized, and a timely follow-up after discharge (Ponikowski et al. Eur Heart J 2016; 37:2129-2200).

LCZ696 is a first-in-class angiotensin receptor neprilysin inhibitor (ARNI) being developed for the treatment of cardiovascular diseases such as hypertension and/or heart failure. Ingestion of LCZ696 results in systemic exposure to sacubitril (AHU377; (2R,4S)-5-biphenyl-4-yl-4-(3-carboxy-propionylamino)-2-methyl-pentanoic acid ethyl ester, also named N-(3-carboxy-1-oxopropyl)-(4S)-(p-phenylphenylmethyl)-4-amino-2R-methylbutanoic acid ethyl ester), a neprilysin (neutral endopeptidase 24.11, NEP) inhibitor (NEPi) prodrug, and valsartan providing inhibition of the angiotensin II type 1 (AT1) receptor, in a 1:1 molar ratio.

Sacubitril is further metabolized via esterases to the active NEPi, LBQ657 (N-(3-carboxy-1-oxopropyl)-(4S)-p-phenylphenylmethyl)-4-amino-(2R)-methylbutanoic acid). Neprilysin degrades biologically active natriuretic peptides (NPs), including atrial natriuretic peptide (ANP), B-type natriuretic peptide (BNP) and C-type natriuretic peptide (CNP). The effects of NEP inhibition are attributed to the enhanced effects of biologically active NPs. NPs, acting through the second messenger cyclic guanosine monophosphate, have potent natriuretic and vasodilator properties, inhibit the activity of the renin-angiotensin-aldosterone system (RAAS), lower sympathetic drive and have anti-fibrotic and anti-hypertrophic effects. Angiotensin receptor blockade is specific and competitive at the angiotensin type 1 (AT1) receptor, which mediates the deleterious effects of angiotensin II on the cardiovascular system. LCZ696, through its dual mode of action, potentiates NPs via NEP inhibition while inhibiting the RAAS via AT1 receptor blockade. Both of these mechanisms are considered to act in a complementary and additive manner to improve the morbidity and mortality of HF patients.

The Prospective comparison of an ARni with an Acei to Determine the Impact on Global Mortality and morbidity in Heart Failure (PARADIGM-HF) trial showed that compared with enalapril, LCZ696 led to a robust 20% relative risk reduction in cardiovascular (CV) mortality and hospitalization for worsening HF among ambulatory HF patients with an ejection fraction (EF)≤40% (ie, changed to ≤35% by an amendment to the protocol midtrial) and New York Heart Association functional class II-IV symptoms (McMurray J J, et al. Eur J Heart Fail 2013; 15:1062-73; McMurray J J, et al. N Engl J Med 2014; 371:993-1004). In response, the American College of Cardiology/American Heart Association/Heart Failure Society of America guidelines were updated in 2016 to recommend as follows: In patients with chronic symptomatic HF with reduced EF, New York Heart Association class II or III, who tolerate an angiotensin converting-enzyme inhibitor (ACEi) or angiotensin receptor blocker (ARB), replacement by an ARNi is recommended to further reduce morbidity and mortality (Yancy C W, et al. 2016, J Am Coll Cardiol 2016; 68:1476-88).

Despite the impressive results seen in the PARADIGM-HF trial, there is limited experience with early initiation of LCZ696 in patients hospitalized for acute decompensated HF (ADHF) and in patients with severe signs and symptoms of HF (McMurray J J, et al. Eur J Heart Fail 2013; 15:1062-73; McMurray J J, et al. N Engl J Med 2014; 371:993-1004).

The TITRATION trial assessed the assess the tolerability of initiating/uptitrating LCZ696 from 50 to 200 mg twice daily (target dose) over 3 and 6 weeks in heart failure (HF) patients (Senni M, et al. Eur J Heart Fail 2016; 18(9):1193-1202).

Thus, HF still represents a major cause of cardiac mortality and morbidity with a clear need for better therapy. Specifically, there is a need for better therapy in patients hospitalized for an ADHF event and diagnosed with a reduced EF. In particular, there is a need for an early initiation of therapy for such patients after being medically stabilized.

### SUMMARY OF THE INVENTION

The present invention relates to a method of treating heart failure with reduced ejection fraction in a patient comprising administering to said patient in need thereof sacubitril and valsartan in a 1:1 molar ratio, wherein treatment is initiated shortly after an acute decompensation heart failure (ADHF) episode of said patient.

The present invention also relates to sacubitril and valsartan in a 1:1 molar ratio for use in a method of treating heart failure with reduced ejection fraction in a patient, wherein treatment is initiated shortly after an acute decompensation heart failure (ADHF) episode of said patient.

The present invention also relates to the use of sacubitril and valsartan in a 1:1 molar ratio in the manufacture of a medicament for treating heart failure with reduced ejection fraction in a patient, wherein treatment is initiated shortly after an acute decompensation heart failure (ADHF) episode of said patient.

The present invention also relates to the use of sacubitril and valsartan in a 1:1 molar ratio for treating heart failure with reduced ejection fraction in a patient, wherein treatment is initiated shortly after an acute decompensation heart failure (ADHF) episode of said patient.

The present invention also relates to a pharmaceutical composition comprising sacubitril and valsartan in a 1:1 molar ratio, and one or more pharmaceutically acceptable carriers, for use in treating heart failure with reduced ejection fraction in a patient, wherein treatment is initiated shortly after an acute decompensation heart failure (ADHF) episode of said patient.

### Definitions

Throughout this specification and in the claims that follow, the following terms are defined with the following meanings, unless explicitly stated otherwise.

The term “treatment” is understood the management and care of a patient for the purpose of combating the disease, condition or disorder.

The term “therapeutically effective amount” refers to an amount of a pharmaceutical composition comprising sacubitril and valsartan in a 1:1 molar ratio that will elicit the desired biological and/or medical response of a tissue, system or an animal (including man) that is being sought by a researcher or clinician.

The term “patient” refers to a human.

The terms “administration of” and or “administering a” compound should be understood to mean providing a pharmaceutical composition comprising sacubitril and valsartan in a 1:1 molar ratio to a subject in need of treatment. The administration of the pharmaceutical composition in order to practice the present invention is carried out by administering a therapeutically effective amount of the pharmaceutical composition to a subject in need of such treatment. The effective amount of the pharmaceutical composition is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgment.

The term “pharmaceutically acceptable”, as used herein, refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

The New York Heart Association (NYHA) classification grades the severity of heart failure symptoms as one of four functional classes. The NYHA classification is widely used in clinical practice and in research because it provides a standard description of severity that can be used to assess response to treatment and to guide management. The New York Heart Association functional classification based on severity of symptoms and physical activity:

Class I: No limitation of physical activity. Ordinary physical activity does not cause undue breathlessness, fatigue, or palpitations.

Class II: Slight limitation of physical activity. Comfortable at rest, but ordinary physical activity results in undue breathlessness, fatigue, or palpitations.

Class III: Marked limitation of physical activity. Comfortable at rest, but less than ordinary physical activity results in undue breathlessness, fatigue, or palpitations.

Class IV: Unable to carry on any physical activity without discomfort. Symptoms at rest can be present. If any physical activity is undertaken, discomfort is increased.

The Child-Pugh score (or its associated Child-Pugh grade) is used as a means to give a description of the clinical state of patients with cirrhosis of the liver, and to indicate the severity of the condition.

The Pugh-Child score is determined by scoring five clinical measures of liver disease. A score of 1, 2, or 3 is given to each measure, with 3 being the most severe.

The five clinical measures are:

(i) total bilirubin: yellow compound in bile from hemoglobin breakdown

(ii) serum albumin: blood protein produced in the liver

(iii) prothrombin time, prolongation(s) or INR: time for blood to clot

(iv) ascites: fluid in peritoneal cavity

(v) hepatic encephalopathy: brain disorder from liver disease

The sum of the five scores from the clinical measures are used to assign a Child-Pugh grade of A (score 5-6), B (score 7-9) or C (score 10-15).

The terms “medically stable” and “hemodynamically stable” are used interchangeably herein. Alternatively, a “medically stable” patient is also referred to as a patient “being stabilized” (after an acute decompensated heart failure episode). These terms characterize a patient as defined by at least one of the following characteristics (i) a systolic blood pressure 100 mm Hg (preferably 110 mm Hg) during 6 hours before initiation of treatment, (ii) no increase (i.e., intensification) in intravenous (IV) diuretics or use of IV vasodilators during 6 hours before initiation of treatment, or (iii) no IV inotropes administered during 24 hours before initiation of treatment.

The term “b.i.d.” as used herein, means twice daily. For example, a dose of 200 mg LCZ696 b.i.d. means that the patient receives twice daily each a 200 mg unit dose of LCZ696, with 400 mg denoting the total daily dose.

The term “acute decompensation heart failure (ADHF) episode” as used herein, refers to a period of medical stabilization after the occurrence of an acute heart failure event wherein the patient receives acute heart failure treatment. Such period is at least 24 hours.

The term “shortly after an acute decompensation heart failure (ADHF) episode” as used herein, refers to a time period starting with medical stabilization after the end of acute heart failure treatment and up to and including 14 days.

### FIGURES

FIG. 1: TRANSITION study design

FIG. 2: Comparison of treatments at baseline for the TRANSITION study, compared to the PARADIGM-HF and TITRATION studies

FIG. 3: Proportion of patients (safety set) meeting the primary and secondary endpoints of the TRANSITION study.

FIG. 4: Most common AEs (2 events in any treatment group) leading to permanent discontinuations (number of patients with at least one AE leading to permanent discontinuation) during the 10-week treatment period in the TRANSITION study.

FIG. 5: Predictors for successful LCZ696 dose up-titration to 200 mg b.i.d in the TRANSITION study.

FIG. 6: Proportion of de novo patients meeting the primary and secondary endpoints of the TRANSITION study.

FIG. 7: Proportion of Arb or ACE naïve patients meeting the primary and secondary endpoints of the TRANSITION study.

FIG. 8: Trial flow diagram of the PIONEER-HF study.

FIG. 9: NT-proBNP by visit change from baseline of geometric mean values in the PIONEER-HF study.

FIG. 10: Percent change from baseline in NT-proBNP geometric mean in the PIONEER-HF study.

FIG. 11: Kaplan-Meier estimated cumulative incidence of the clinical composite of death from any cause, hospitalization for worsening HF, left ventricular assist device implantation, or listing for cardiac transplant in the PIONEER-HF study.

FIG. 12: Subgroup analyses of change in NT-proBNP in evaluable patients by age, sex, race, prior HF and NYHA class, SBP, or NT-proBNP values, LVEF, eGFR, atrial fibrillation, hypertension, time from admission to randomization and prior ACEi and/or ARB use in the PIONEER-HF study.

FIG. 13: Subgroup analyses of clinical composite of death from any cause, hospitalization for worsening HF, left ventricular assist device implantation, or listing for cardiac transplantation by age, sex, race, prior HF and NYHA class, SBP, or NT-proBNP values, LVEF, eGFR, atrial fibrillation, hypertension, time from admission to randomization and prior ACEi and/or ARB use in the PIONEER-HF study.

### DETAILED DESCRIPTION OF THE INVENTION

This invention is based on the clinical trials TRANSITION (NCT02661217) and PIONEER-HF (NCT02554890).

The clinical trial TRANSITION (NCT02661217) has compared Pre-discharge and Post-discharge treatment initiation with LCZ696 therapy in heart failure patients with reduced ejection fraction (HFrEF) shortly after an acute decompensation heart failure (ADHF) episode. It has been shown that
Comparable proportions of patients met the primary and secondary endpoints in the pre- and post-discharge initiation groups.
The incidence of adverse events and discontinuations due to AEs was similar in in-hospital and ambulatory initiation groups.
About half of HFrEF patients stabilized after an acute HF decompensation event achieved within 10 weeks the target dose of 200 mg LCZ696 b.i.d.
Early initiation of LCZ696 was feasible and overall well tolerated
   in wide range of HFrEF patients, including de novo HF and ACEi/ARB naïve patients;
   stabilized HFrEF patients shortly after an ADHF episode;
   was not associated with any new types of adverse events when compared to the PARADIGM-HF and TITRATION patient populations; and
   in-line with safety profile of other disease-modifying HF therapies.

The clinical trial PIONEER-HF (NCT02554890) was designed to assess the effect of in-hospital initiation of LCZ696 versus enalapril on the time-averaged proportional change in NT-proBNP levels from baseline to Weeks 4 and 8 in hemodynamically stable patients with HFrEF (LVEF 40%) following hospitalization for ADHF. It has been shown that
Treatment with LCZ696 compared with enalapril, initiated after initial stabilization after an ADHF episode, was well tolerated and led to early and sustained reduction in NT-proBNP concentration notable already by the first week. Compared with enalapril, LCZ696 led to an increased reduction in the clinical composite of death, rehospitalization for HF, LVAD implantation, or listing for cardiac transplant over the 8-week study period, with relative and absolute risk reductions with sacubitril/valsartan compared with enalapril of 44.6% and 7.5%, respectively.
The existing evidence-base for ARNi (i.e. LCZ696) therapy can be extended to populations previously not studied, including patients hospitalized for ADHF with de novo HF not already known to tolerate high doses of guideline-directed HF medications or not receiving conventional renin-angiotensin system inhibitors (Ambrosy A P, et al. Eur J Heart Fail 2018; 20:963-72).
LCZ696 was more effective than enalapril in a patient population of patients which identified semselves as black and for whom evidence of ARNi therapy from prior clinical studies was limited. Also in this patient population, it could be shown that there were no angioedema with LCZ696, whereas angioedema were observed with enalapril.

Taken both studies together, it has been shown that LCZ696 can be safely initiated shortly after an acute heart failure episode, both in-hospital and in an out-patient setting and in a wide range of HFrEF patients.

The target dose of 200 mg LCZ696 b.i.d. is the dose studied in the PARADIGM-HF trial that demonstrated superiority over enalapril 10 mg b.i.d. A dose of 200 mg LCZ696 b.i.d. delivers similar exposures of valsartan (assessed by AUC) as valsartan 160 mg b.i.d., the maximal approved valsartan dose for HF and the dose recommended in international guidelines for the treatment of HF. In addition, biomarker analysis (increase in levels of ANP and cGMP) indicated that this dose delivers approximately 90% of its maximal neprilysin inhibition (Gu J, et al. J Clin Pharmacol 2010; 50:401-14).

The achievement of the target dose is indicative of a patient's tolerability to the treatment, since the basis for up-titration towards the target dose was based on tolerability (i.e., incidence of hypotension, renal or hepatic impairment and hyperkalemia) based on clinical evaluation and laboratory assessment of the patient. For example, the high percentage of patients reaching the target dose of 200 mg LCZ696 b.i.d. in the TRANSITION study proves that early initiation of LCZ696 was feasible and overall well tolerated.

Prior trial evidence for ARNi in HFrEF was limited to patients who were on established, stable high doses of an ACEi/ARB and who tolerated sequential single-blind run-in periods to document tolerability of the highest dose of enalapril and sacubitril/valsartan prior to randomization. The PIONEER-HF study made use of the lowest dose of sacubitril/valsartan (24/26 mg tablet), which was not previously tested. Using an initial dose selection and further dose escalation following a SBP-based algorithm, patients enrolled in PIONEER-HF safely achieved the SBP-based target dose of sacubitril/valsartan. Importantly, the favorable effects seen with sacubitril/valsartan did not differ based on the SBP at baseline, ACEi/ARB use at hospital admission, prior diagnosis of HF, race, time from symptom presentation.

In the context of the present invention, the term “sacubitril and valsartan in a 1:1 molar ratio” refers to a combination of a 1:1 molar ratio of
(i) valsartan or a pharmaceutically acceptable salt thereof; and
(ii) sacubitril or a pharmaceutically acceptable salt thereof.

In one embodiment, said combination is provided in form of a complex or compound comprising valsartan and sacubitril and linking them together via non-covalent or covalent bonding, optionally via a linker.

In one embodiment, sacubitril and valsartan in a 1:1 molar ratio is provided in the form of the compound of the formula (I)

[(A₁)(A₂)](Na⁺)₃.xH₂O (I)

wherein
A₁ is valsartan in the dianionic form;
A₂ is sacubitril in the anionic form;
Na⁺ is a sodium ion; and
x is 0.5 to 7.

In one embodiment thereof, sacubitril and valsartan in a 1:1 molar ratio is provided in the form of the compound of the formula (I), wherein x is 0.5 to 3.5.

In one embodiment thereof, sacubitril and valsartan in a 1:1 molar ratio is provided in the form of the compound of the formula (I), wherein x is 0.5 to 2.5.

In one embodiment thereof, sacubitril and valsartan in a 1:1 molar ratio is provided in the form of the compound of the formula (I), wherein x is 2.5 to 3.5.

In one embodiment thereof, the compound of formula (I) is in amorphous form.

In one embodiment thereof, sacubitril and valsartan in a 1:1 molar ratio is provided in the form of the compound of the formula (I), wherein x is 2.5.

In one embodiment thereof, the compound of formula (I) is in crystalline form.

In one embodiment thereof, the compound of formula (I) is trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate.

In one embodiment thereof, the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate is present in crystalline form.

In the context of the present invention, a “pharmaceutical composition for use” is a pharmaceutical composition comprising sacubitril and valsartan in a 1:1 molar ratio, wherein sacubitril and valsartan in a 1:1 molar ratio is as defined in the embodiments above.

Pharmaceutical compositions and compounds containing sacubitril and valsartan, such as LCZ696, and their uses have for example been previously disclosed in WO2003059345, WO2007056546, WO2009061713, WO2012027237, WO2014029848, WO2015030711, WO2015028941, WO2016181284, WO2016193883, WO2017006254 and WO2017037577, which are herein incorporated by reference.

Pharmaceutical compositions and compounds containing sacubitril and valsartan in a 1:1 molar ratio and their uses have also for example been previously disclosed in CN105037289A, WO2017096772, WO2016037552, WO2016049663, CN105461647A, WO2016051393, CN105503760A, CN105669581A, WO2016125123, CN105929031A, WO2016151525, CN106032361A, WO2016201238, CN106316973A, WO2017012917, WO2017009784, WO2017037591, WO2017036420, WO2017037596, WO2017042700, CN106518709A, WO2017085573, IN03835DE2015, WO17097085, IN04304DE2015, CN107033094A, WO2017154017, WO2017191619, IN201641010897A, IN201641022870A, CN107674038A, TW201806936A, WO2018069833, WO2018069937, and WO2018078592, which are herein incorporated by reference.

(i) Valsartan or (S)-N-valeryl-N-{[2′-(1H-tetrazole-5-yl)-biphenyl-4-yl]-methyl}-valine) or a pharmaceutically acceptable salt thereof that can be purchased from commercial sources or can be prepared according to known methods, such as described in U.S. Pat. No. 5,399,578 and EP 0443983, whose preparative teachings are incorporated by reference herein. Valsartan may be used in certain embodiments of the invention in its free acid form, as well as in any suitable salt form. Depending upon the circumstance, esters or other derivatives of the carboxylic grouping may be employed as well as salts and derivatives of the tetrazole grouping.

(ii) Sacubitril or N-(3-carboxy-1-oxopropyl)-(4S)-(p-phenylphenylmethyl)-4-amino-2R-methylbutanoic acid ethyl ester or a pharmaceutically acceptable salt thereof as well as (2R,4S)-5-biphenyl-4-yl-4(3-carboxy-propionyl amino)-2-methyl-pentanoic acid can be prepared by known methods such as described in U.S. Pat. No. 5,217,996 which is herein incorporated by reference.

The corresponding active ingredients or pharmaceutically acceptable salts thereof may also be used in the form of a hydrate or include other solvents used for crystallization.

Preferably, the compounds sacubitril or a salt thereof, valsartan or a salt thereof, compounds of formula (I), in particular LCZ696, are substantially pure or in a substantially pure form. As used herein, “substantially pure” refers to at least about 90% purity, more preferably at least about 95% and most preferably at least about 98% purity.

Also preferred is that these compounds are solid or in a solid form or in solid state. The solid, solid form or solid state can be crystalline, partially crystalline, amorphous or poly-amorphous, preferably in the crystalline form.

The pharmaceutical compositions can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carriers, especially suitable for enteral or parenteral application.

The pharmaceutical compositions contain, for example, from about 0.1% to about 100%, e.g. 80% or 90%, or from about 1% to about 60%, of the active ingredient. The term “about” or “approximately”, as used herein in each instance, shall have the meaning of within 10%, more preferably within 5%, of a given value or range.

Pharmaceutical compositions for enteral or parenteral administration are, e.g., those in unit dose forms, such as sugar-coated tablets, tablets, capsules, bars, sachets, granules, syrups, aqueous or oily suspensions or suppositories and furthermore ampoules. These are prepared in a manner known per se, e.g. by means of conventional mixing, granulating, sugar-coating, dissolving or lyophilizing processes. Thus, pharmaceutical compositions for oral use can be obtained by combining the active ingredient with solid carriers, if desired granulating a mixture obtained, and processing the mixture or granules, if desired or necessary, after addition of suitable excipients to give tablets or sugar-coated tablet cores.

Tablets may be formed from the active compound with fillers, for example calcium phosphate; disintegrating agents, for example maize starch, lubricating agents, for example magnesium stearate; binders, for example microcrystalline cellulose or polyvinylpyrrolidone and other optional ingredients known in the art to permit tabletting the mixture by known methods. Similarly, capsules, for example hard or soft gelatin capsules, containing the active compound with or without added excipients, may be prepared by known methods. The contents of the capsule may be formulated using known methods so as to give sustained release of the active compound.

Other dosage forms for oral administration include, for example, aqueous suspensions containing the active compound in an aqueous medium in the presence of a non-toxic suspending agent such as sodium carboxymethylcellulose, and oily suspensions containing the active compounds in a suitable vegetable oil, for example arachis oil.

The pharmaceutical compositions include active compounds that are formulated into granules with or without additional excipients. The granules may be ingested directly by the patient or they may be added to a suitable liquid carrier (e.g. water) before ingestion.

The granules may contain disintegrants, e.g. an effervescent pair formed from an acid and a carbonate or bicarbonate salt to facilitate dispersion in the liquid medium.

The dosage of the active ingredients in the composition will vary with the nature of and the severity of the condition to be treated and with the particular active ingredient or active ingredients in the composition and its route of administration. It will also vary according to the age, weight and response of the individual patient.

In one embodiment, the combined unit dose of the therapeutic agents sacubitril and valsartan together in the pharmaceutical composition will be in the range from about 1 to about 1000 mg, such as 40 mg to 400 mg (e.g., 50 mg, 100 mg, 200 mg, 400 mg). Alternatively, pharmaceutical compositions with lower doses may be prepared, for example combined unit doses of 0.5 to 100 mg; 0.5 to 50 mg; or 0.5 to 20 mg of sacubitril and valsartan. In embodiments, where sacubitril and valsartan in a 1:1 molar ratio are presented in the form of trisodium [3-((1S,3R)-1-biphenyl-4-ylmethyl-3-ethoxycarbonyl-1-butylcarbamoyl) propionate-(S)-3′-methyl-2′-(pentanoyl{2″-(tetrazol-5-ylate)biphenyl-4′-ylmethyl}amino)butyrate] hemipentahydrate, a unit dose of for example 100 mg LCZ696 delivers 100 mg of the two agents sacubitril and valsartan (i.e. 49 mg sacubitril and 51 mg valsartan) and amounts to 113.1 mg of trisodium [3-((1S,3R)-1-biphenyl-4-ylmethyl-3-ethoxycarbonyl-1-butylcarbamoyl) propionate-(S)-3′-methyl-2′-(pentanoyl{2″-(tetrazol-5-ylate)biphenyl-4′-ylmethyl}amino)butyrate]hemipentahydrate. Correspondingly, a unit dose of 50 mg LCZ696 requires 56.6 mg, a unit dose of 200 mg LCZ696 requires 226.2 mg, and a unit dose of 400 mg LCZ696 requires 452.4 mg of trisodium [3-((1S,3R)-1-biphenyl-4-ylmethyl-3-ethoxycarbonyl-1-butylcarbamoyl) propionate-(S)-3′-methyl-2′-(pentanoyl{2″-(tetrazol-5-ylate)biphenyl-4′-ylmethyl}amino)butyrate]hemipentahydrate, respectively.

Pharmaceutical compositions as used in the current invention can be administered any number of times per day, i.e. once a day (q.d.), twice (b.i.d.), three times, four time, etc. in an immediate release formation or less frequently as an extended or sustained release formation. Preferably the pharmaceutical composition is administered twice daily (b.i.d.). Corresponding doses may be taken, for example, in the morning, at mid-day or in the evening.

In one embodiment of the present invention, the pharmaceutical composition is administered to deliver a daily overall dose of the combination of sacubitril and valsartan in a 1:1 molar ratio from about 50 mg to about 1000 mg, in particular to about 400 mg, or to about 200 mg.

In one embodiment thereof, the pharmaceutical composition is administered to deliver the combination of sacubitril and valsartan in a 1:1 molar ratio twice daily (b.i.d.) with a dose of 50 mg, 100 mg, or 200 mg. in other words, the combination of sacubitril and valsartan in a 1:1 molar ratio is administered to the patient twice daily with an individual dose of 50 mg, 100 mg, or 200 mg, totaling to a daily dose of 100 mg, 200 mg or 400 mg, respectively.

In one embodiment thereof,
a) the 50 mg dose of sacubitril and valsartan in a 1:1 molar ratio corresponds to 24 mg sacubitril and 26 mg valsartan,
b) the 100 mg dose of sacubitril and valsartan in a 1:1 molar ratio corresponds to 49 mg sacubitril and 51 mg valsartan, and
c) the 200 mg dose of sacubitril and valsartan in a 1:1 molar ratio corresponds to 97 mg sacubitril and 103 mg valsartan.

In a particular embodiment of the pharmaceutical composition, the combination of sacubitril and valsartan in a 1:1 molar ratio is delivered in the form of the compound trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate, wherein
a) the 50 mg dose of sacubitril and valsartan in a 1:1 molar ratio corresponds to around 56.6 mg trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate,
b) the 100 mg dose of sacubitril and valsartan in a 1:1 molar ratio corresponds to around 113.1 mg trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate, and
c) the 200 mg dose of sacubitril and valsartan in a 1:1 molar ratio corresponds to around 226.2 mg trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate.

Accordingly, the present invention relates to the following embodiments:

### METHOD OF TREATMENT EMBODIMENTS

### Embodiment 1

A method of treating heart failure with reduced ejection fraction in a patient comprising administering to said patient in need thereof sacubitril and valsartan in a 1:1 molar ratio, wherein treatment is initiated shortly after an acute decompensation heart failure episode of said patient, wherein the term shortly refers to a time period starting with medical stabilization after the end of acute heart failure treatment and up to and including 14 days after an acute decompensation heart failure episode.

### Embodiment 2

A method according to Embodiment 1, wherein the treatment is initiated while the patient is still hospitalized due to the acute decompensation heart failure episode.

### Embodiment 3

A method according to Embodiment 1 or 2, wherein the treatment is initiated within 2 weeks after an acute decompensation heart failure episode.

### Embodiment 4

A method according to Embodiment 1 or 2, wherein the treatment is initiated within 10 days after an acute decompensation heart failure episode.

### Embodiment 5

A method according to any one of Embodiments 1 to 4, wherein the patient is hemodynamically stable.

### Embodiment 6

A method according to Embodiment 5, wherein the hemodynamically stable patient is characterized by at least one of the following (i) a systolic blood pressure 100 mm Hg during 6 hours before initiation of treatment, (ii) no increase in IV diuretics or use of IV vasodilators during 6 hours before initiation of treatment, or (iii) no IV inotropes administered during 24 hours before initiation of treatment.

### Embodiment 7

A method according to Embodiment 6, wherein the hemodynamically stable patient is characterized by (i) a systolic blood pressure 100 mm Hg during 6 hours before initiation of treatment, (ii) no increase in IV diuretics or use of IV vasodilators during 6 hours before initiation of treatment, and (iii) no IV inotropes administered during 24 hours before initiation of treatment.

### Embodiment 8

A method according any one of Embodiments 1 to 7, wherein patient suffers from heart failure with reduced ejection fraction based on at least one of the following characteristics
i) heart failure of NYHA class II, III or IV,
ii) an elevated plasma BNP or NT-proBNP level, preferably a plasma BNP≥100 pg/mL or NT-proBNP≥400 pg/mL, more preferably a plasma BNP≤150 pg/mL or NT-proBNP≥600 pg/mL, even more preferably a plasma BNP≥450 pg/mL or NT-proBNP≥1600 pg/mL, or
iii) a reduced left ventricular ejection fraction (LVEF) of ≤40%.

### Embodiment 9

A method according to Embodiment 8, wherein the patient suffers from heart failure with reduced ejection fraction classified as NYHA class II, III or IV and wherein the patient has a reduced left ventricular ejection fraction (LVEF) of ≤40%.

### Embodiment 10

A method according to any one of Embodiments 1 to 9, wherein the patient has been diagnosed as suffering from heart failure with reduced ejection fraction prior to said acute decompensation heart failure episode mentioned in Embodiment 1.

### Embodiment 11

A method according to any one of Embodiments 1 to 9, wherein the patient is a de novo patient not having been diagnosed as suffering from heart failure with reduced ejection fraction prior to said acute decompensation heart failure episode mentioned in Embodiment 1.

### Embodiment 12

A method according to any one of Embodiments 1 to 11, wherein the patient has not received an ACEI or ARB or both for at least 4 weeks prior to said acute decompensation heart failure episode mentioned in Embodiment 1.

### Embodiment 13

A method according to any one of Embodiments 1 to 11, wherein the patient is an ACEI/ARB naïve patient not having received an ACEI or ARB or both prior to said acute decompensation heart failure episode mentioned in Embodiment 1.

### Embodiment 14

A method according to any one of Embodiments 1 to 13, wherein the patient achieves a target dose of 200 mg of sacubitril and valsartan in a 1:1 molar ratio b.i.d.

### Embodiment 15

A method according to any one of Embodiments 1 to 13, wherein the patient achieves a target dose of 100 mg of sacubitril and valsartan in a 1:1 molar ratio b.i.d.

### Embodiment 16

A method according to any one of Embodiments 1 to 13, wherein the patient achieves a target dose of 50 mg of sacubitril and valsartan in a 1:1 molar ratio b.i.d.

### Embodiment 17

A method according to any one of Embodiments 14 to 16, wherein the target dose is reached after titration from a starting dose of sacubitril and valsartan in a 1:1 molar ratio b.i.d. increasing to the target dose during an up-titration period from about 2 to about 10 weeks.

### Embodiment 18

A method according to Embodiments 14 or 15, wherein the target dose is reached after titration from a starting dose of 50 mg of sacubitril and valsartan in a 1:1 molar ratio b.i.d. increasing to the target dose during an up-titration period of from about 2 to about 10 weeks.

### Embodiment 19

A method according to Embodiment 18, wherein the starting dose of 50 mg of sacubitril and valsartan in a 1:1 molar ratio b.i.d. is used in a patient not taking an ACEI or ARB before initiation of treatment.

### Embodiment 20

A method according to Embodiment 18, wherein the starting dose of 50 mg of sacubitril and valsartan in a 1:1 molar ratio b.i.d. is used in a patient taking low doses of an ACEI or ARB before initiation of treatment.

### Embodiment 21

A method according to Embodiment 18, wherein the starting dose of 50 mg of sacubitril and valsartan in a 1:1 molar ratio b.i.d. is used in a patient with moderate hepatic impairment (Child-Pugh grade B classification), or with AST/ALT values more than twice the upper limit of the normal range before initiation of treatment.

### Embodiment 22

A method according to Embodiment 18, wherein the starting dose of 50 mg of sacubitril and valsartan in a 1:1 molar ratio b.i.d. is used in a patient with moderate renal impairment (eGFR 30-60 ml/min/1.73 m²) before initiation of treatment.

### Embodiment 23

A method according to Embodiment 14, wherein the target dose is reached after a titration from a starting dose of 100 mg of sacubitril and valsartan in a 1:1 molar ratio b.i.d. increasing to the target dose during an up-titration period of from about 2 to about 10 weeks.

### Embodiment 24

A method according to any one of Embodiments 17 to 23, wherein the up-titration period is from about 2 to about 8 weeks.

### Embodiment 25

A method according to Embodiment 24, wherein the up-titration period is from about 2 to about 6 weeks.

### Embodiment 26

A method according to Embodiment 25, wherein the up-titration period is from about 2 to about 4 weeks.

### Embodiment 27

A method according to any one of Embodiments 1 to 26, wherein sacubitril and valsartan in a 1:1 molar ratio refers to a combination of a 1:1 molar ratio of
(i) valsartan or a pharmaceutically acceptable salt thereof; and
(ii) sacubitril or a pharmaceutically acceptable salt thereof.

### Embodiment 28

A method according to Embodiment 27, wherein sacubitril and valsartan in a 1:1 molar ratio is provided in the form of the compound of the formula (I)

[(A₁)(A₂)](Na⁺)₃.xH₂O (I)

wherein
A₁ is valsartan in the dianionic form;
A₂ is sacubitril in the anionic form;
Na⁺ is a sodium ion; and
x is 0.5 to 7.

### Embodiment 29

A method according to Embodiment 27 or 28, wherein sacubitril and valsartan in a 1:1 molar ratio is provided in the form of the compound of the formula (I), wherein x is 0.5 to 3.5.

### Embodiment 30

A method according to any one of Embodiments 27 to 29, wherein sacubitril and valsartan in a 1:1 molar ratio is provided in the form of the compound of the formula (I), wherein x is 2.5.

### Embodiment 31

A method according to any one of Embodiments 27 to 30, wherein sacubitril and valsartan in a 1:1 molar ratio is provided in the form of the compound of the formula (I), which is trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate.

### Embodiment 32

A method according to any one of Embodiments 1 to 31, wherein the patient receives a base treatment with a stable dose of a beta-blocker, an aldosterone antagonists, and/or a diuretic.

### Embodiment 33

A method according to any one of Embodiments 1 to 31, wherein sacubitril and valsartan in a 1:1 molar ratio has been shown to reduce the clinical composite endpoint of death, rehospitalization for HF, LVAD implantation, or listing for cardiac transplant.

### Embodiment 34

A method according to any one of Embodiments 1 to 33, wherein sacubitril and valsartan in a 1:1 molar ratio is more effective than a medicament comprising a therapeutically effective amount of an ACE inhibitor, preferably wherein the ACE inhibitor is enalapril.

### Embodiment 35

A method according to Embodiment 34, wherein sacubitril and valsartan in a 1:1 molar ratio is at least 10%, preferably at least 15%, preferably at least 20%, more effective than a medicament comprising a therapeutically effective amount of enalapril in reducing the clinical composite endpoint of death, rehospitalization for HF, LVAD implantation, or listing for cardiac transplant.

### Embodiment 36

A method according to Embodiment 34, wherein sacubitril and valsartan in a 1:1 molar ratio has been shown to be statistically superior to a medicament comprising a therapeutically effective amount of enalapril in reducing the clinical composite endpoint of death, rehospitalization for HF, LVAD implantation, or listing for cardiac transplant.

### Embodiment 37

A method according to Embodiment 36, wherein the statistical superiority is expressed as a relative risk reduction rate of sacubitril and valsartan in a 1:1 molar ratio compared with enalapril of at least 40%.

### Embodiment 38

A method according to Embodiment 36, wherein the statistical superiority is expressed as an absolute risk reduction rate of sacubitril and valsartan in a 1:1 molar ratio compared with enalapril of at least 5%, preferably at least 7%.

### SACUBITRIL AND VALSARTAN FOR USE EMBODIMENTS

### Embodiment 1b

Sacubitril and valsartan in a 1:1 molar ratio for use in a method of treating heart failure with reduced ejection fraction in a patient comprising, wherein treatment is initiated shortly after an acute decompensation heart failure episode of said patient, wherein the term shortly refers to a time period starting with medical stabilization after the end of acute heart failure treatment and up to and including 14 days after an acute decompensation heart failure episode.

### Embodiment 2b

Sacubitril and valsartan in a 1:1 molar ratio for use according to Embodiment 1b, wherein the treatment is initiated while the patient is still hospitalized due to the acute decompensation heart failure episode.

### Embodiment 3b

Sacubitril and valsartan in a 1:1 molar ratio for use according to Embodiment 1b or 2b, wherein the treatment is initiated within 2 weeks after an acute decompensation heart failure episode.

### Embodiment 4b

Sacubitril and valsartan in a 1:1 molar ratio for use according to Embodiment 1b or 2b, wherein the treatment is initiated within 10 days after an acute decompensation heart failure episode.

### Embodiment 5b

Sacubitril and valsartan in a 1:1 molar ratio for use according to any one of Embodiments 1b to 4b, wherein the patient is hemodynamically stable.

### Embodiment 6b

Sacubitril and valsartan in a 1:1 molar ratio for use according to Embodiment 5b, wherein the hemodynamically stable patient is characterized by at least one of the following (i) a systolic blood pressure 100 mm Hg during 6 hours before initiation of treatment, (ii) no increase in IV diuretics or use of IV vasodilators during 6 hours before initiation of treatment, or (iii) no IV inotropes administered during 24 hours before initiation of treatment.

### Embodiment 7b

Sacubitril and valsartan in a 1:1 molar ratio for use according to Embodiment 6b, wherein the hemodynamically stable patient is characterized by (i) a systolic blood pressure 100 mm Hg during 6 hours before initiation of treatment, (ii) no increase in IV diuretics or use of IV vasodilators during 6 hours before initiation of treatment, and (iii) no IV inotropes administered during 24 hours before initiation of treatment.

### Embodiment 8b

Sacubitril and valsartan in a 1:1 molar ratio for use according any one of Embodiments 1b to 7b, wherein patient suffers from heart failure based with reduced ejection fraction on at least one of the following characteristics
i) heart failure of NYHA class II, III or IV,
ii) an elevated plasma BNP or NT-proBNP level, preferably a plasma BNP≥100 pg/mL or NT-proBNP≥400 pg/mL, more preferably a plasma BNP≥150 pg/mL or NT-proBNP≥600 pg/mL, even more preferably a plasma BNP≥450 pg/mL or NT-proBNP≥1600 pg/mL, or
iii) a reduced left ventricular ejection fraction (LVEF) of ≤40%.

### Embodiment 9b

Sacubitril and valsartan in a 1:1 molar ratio for use according to Embodiment 8b, wherein the patient suffers from heart failure with reduced ejection fraction classified as NYHA class II, III or IV and has a reduced left ventricular ejection fraction (LVEF) of ≤40%.

### Embodiment 10b

Sacubitril and valsartan in a 1:1 molar ratio for use according to any one of Embodiments 1b to 9b, wherein the patient has been diagnosed as suffering from heart failure with reduced ejection fraction prior to said acute decompensation heart failure episode mentioned in Embodiment 1b.

### Embodiment 11b

Sacubitril and valsartan in a 1:1 molar ratio for use according to any one of Embodiments 1b to 9b, wherein the patient is a de novo patient not having been diagnosed as suffering from heart failure with reduced ejection fraction prior to said acute decompensation heart failure episode mentioned in Embodiment 1b.

### Embodiment 12b

Sacubitril and valsartan in a 1:1 molar ratio for use according to any one of Embodiments 1b to 11b, wherein the patient has not received an ACEI or ARB or both for at least 4 weeks prior to said acute decompensation heart failure episode mentioned in Embodiment 1b.

### Embodiment 13b

Sacubitril and valsartan in a 1:1 molar ratio for use according to any one of Embodiments 1b to 11b, wherein the patient is an ACEI/ARB naïve patient not having received an ACEI or ARB or both prior to said acute decompensation heart failure episode mentioned in Embodiment 1b.

### Embodiment 14b

Sacubitril and valsartan in a 1:1 molar ratio for use according to any one of Embodiments 1b to 13b, wherein the patient achieves a target dose of 200 mg of sacubitril and valsartan in a 1:1 molar ratio b.i.d.

### Embodiment 15b

Sacubitril and valsartan in a 1:1 molar ratio for use according to any one of Embodiments 1b to 13b, wherein the patient achieves a target dose of 100 mg of sacubitril and valsartan in a 1:1 molar ratio b.i.d.

### Embodiment 16b

Sacubitril and valsartan in a 1:1 molar ratio for use according to any one of Embodiments 1b to 13b, wherein the patient achieves a target dose of 50 mg of sacubitril and valsartan in a 1:1 molar ratio b.i.d.

### Embodiment 17b

Sacubitril and valsartan in a 1:1 molar ratio for use according to any one of Embodiments 14b to 16b, wherein the target dose is reached after titration from a starting dose of sacubitril and valsartan in a 1:1 molar ratio b.i.d. increasing to the target dose during an up-titration period from about 2 to about 10 weeks.

### Embodiment 18b

Sacubitril and valsartan in a 1:1 molar ratio for use according to Embodiments 14b or 15b, wherein the target dose is reached after titration from a starting dose of 50 mg of sacubitril and valsartan in a 1:1 molar ratio b.i.d. increasing to the target dose during an up-titration period of from about 2 to about 10 weeks.

### Embodiment 19b

Sacubitril and valsartan in a 1:1 molar ratio for use according to Embodiment 18b, wherein the starting dose of 50 mg of sacubitril and valsartan in a 1:1 molar ratio b.i.d. is used in a patient not taking an ACEI or ARB before initiation of treatment.

### Embodiment 20b

Sacubitril and valsartan in a 1:1 molar ratio for use according to Embodiment 18b, wherein the starting dose of 50 mg of sacubitril and valsartan in a 1:1 molar ratio b.i.d. is used in a patient taking low doses of an ACEI or ARB before initiation of treatment.

### Embodiment 21b

Sacubitril and valsartan in a 1:1 molar ratio for use according to Embodiment 18b, wherein the starting dose of 50 mg of sacubitril and valsartan in a 1:1 molar ratio b.i.d. is used in a patient with moderate hepatic impairment (Child-Pugh grade B classification), or with AST/ALT values more than twice the upper limit of the normal range before initiation of treatment.

### Embodiment 22b

Sacubitril and valsartan in a 1:1 molar ratio for use according to Embodiment 18b, wherein the starting dose of 50 mg of sacubitril and valsartan in a 1:1 molar ratio b.i.d. is used in a patient with moderate renal impairment (eGFR 30-60 ml/min/1.73 m²) before initiation of treatment.

### Embodiment 23b

Sacubitril and valsartan in a 1:1 molar ratio for use according to Embodiment 14b, wherein the target dose is reached after a titration from a starting dose of 100 mg of sacubitril and valsartan in a 1:1 molar ratio b.i.d. increasing to the target dose during an up-titration period of from about 2 to about 10 weeks.

### Embodiment 24b

Sacubitril and valsartan in a 1:1 molar ratio for use according to any one of Embodiments 17b to 23b, wherein the up-titration period is from about 2 to about 8 weeks.

### Embodiment 25b

Sacubitril and valsartan in a 1:1 molar ratio for use according to Embodiment 24b, wherein the up-titration period is from about 2 to about 6 weeks.

### Embodiment 26b

Sacubitril and valsartan in a 1:1 molar ratio for use according to Embodiment 25b, wherein the up-titration period is from about 2 to about 4 weeks.

### Embodiment 27b

Sacubitril and valsartan in a 1:1 molar ratio for use according to any one of Embodiments 1b to 26b, wherein sacubitril and valsartan in a 1:1 molar ratio refers to a combination of a 1:1 molar ratio of
(i) valsartan or a pharmaceutically acceptable salt thereof; and
(ii) sacubitril or a pharmaceutically acceptable salt thereof.

### Embodiment 28b

Sacubitril and valsartan in a 1:1 molar ratio for use according to Embodiment 27b, wherein sacubitril and valsartan in a 1:1 molar ratio is provided in the form of the compound of the formula (I)

[(A₁)(A₂)](Na⁺)₃.xH₂O (I)

wherein
A₁ is valsartan in the dianionic form;
A₂ is sacubitril in the anionic form;
Na⁺ is a sodium ion; and
x is 0.5 to 7.

### Embodiment 29b

Sacubitril and valsartan in a 1:1 molar ratio for use according to Embodiment 27b or 28b, wherein sacubitril and valsartan in a 1:1 molar ratio is provided in the form of the compound of the formula (I), wherein x is 0.5 to 3.5.

### Embodiment 30b

Sacubitril and valsartan in a 1:1 molar ratio for use according to any one of Embodiments 27b to 29b, wherein sacubitril and valsartan in a 1:1 molar ratio is provided in the form of the compound of the formula (I), wherein x is 2.5.

### Embodiment 31b

Sacubitril and valsartan in a 1:1 molar ratio for use according to any one of Embodiments 27b to 30b, wherein sacubitril and valsartan in a 1:1 molar ratio is provided in the form of the compound of the formula (I), which is trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate.

### Embodiment 32b

Sacubitril and valsartan in a 1:1 molar ratio for use according to any one of Embodiments 1b to 31b, wherein the patient receives a base treatment with a stable dose of a beta-blocker, an aldosterone antagonists, and/or a diuretic.

### Embodiment 33b

Sacubitril and valsartan in a 1:1 molar ratio for use according to any one of Embodiments 1b to 31b, wherein sacubitril and valsartan in a 1:1 molar ratio has been shown to reduce the clinical composite endpoint of death, rehospitalization for HF, LVAD implantation, or listing for cardiac transplant.

### Embodiment 34b

Sacubitril and valsartan in a 1:1 molar ratio for use according to any one of Embodiments 1b to 33b, wherein sacubitril and valsartan in a 1:1 molar ratio is more effective than a medicament comprising a therapeutically effective amount of an ACE inhibitor, preferably wherein the ACE inhibitor is enalapril.

### Embodiment 35b

Sacubitril and valsartan in a 1:1 molar ratio for use according to Embodiment 34b, wherein sacubitril and valsartan in a 1:1 molar ratio is at least 10%, preferably at least 15%, preferably at least 20%, more effective than a medicament comprising a therapeutically effective amount of enalapril in reducing the clinical composite endpoint of death, rehospitalization for HF, LVAD implantation, or listing for cardiac transplant.

### Embodiment 36b

Sacubitril and valsartan in a 1:1 molar ratio for use according to Embodiment 34b, wherein sacubitril and valsartan in a 1:1 molar ratio has been shown to be statistically superior to a medicament comprising a therapeutically effective amount of enalapril in reducing the clinical composite endpoint of death, rehospitalization for HF, LVAD implantation, or listing for cardiac transplant.

### Embodiment 37b

Sacubitril and valsartan in a 1:1 molar ratio for use according to Embodiment 36b, wherein the statistical superiority is expressed as a relative risk reduction rate of sacubitril and valsartan in a 1:1 molar ratio compared with enalapril of at least 40%.

### Embodiment 38b

Sacubitril and valsartan in a 1:1 molar ratio for use according to Embodiment 36b, wherein the statistical superiority is expressed as an absolute risk reduction rate of sacubitril and valsartan in a 1:1 molar ratio compared with enalapril of at least 5%, preferably at least 7%.

All the aforementioned enumerated embodiments relating to the methods of treatment or to sacubitril and valsartan in a 1:1 molar ratio for use in the treatment according to the present invention are equally applicable to
use of sacubitril and valsartan in a 1:1 molar ratio in the manufacture of a medicament for the treatment according to the present invention,
the use of sacubitril and valsartan in a 1:1 molar ratio for the treatment according to the present invention,
a pharmaceutical composition comprising sacubitril and valsartan in a 1:1 molar ratio, and one or more pharmaceutically acceptable carriers, for use in the treatment according to the present invention.

### EXAMPLES

### Abbreviations

| | |
|---|---|
| ACEi, ACEI | angiotensin-converting-enzyme inhibitor |
| ADHF | acute decompensated heart failure |
| ARB | angiotensin II receptor blocker |
| b.i.d | twice daily |
| h | hour |
| OMT | optimized medical treatment |
| ARNi, ARNI | angiotensin receptor neprilysin inhibitor |
| BNP | B-type natriuretic peptide |
| eGFR | estimated glomerular filtration rate |
| HF | heart failure |
| LVEF | left ventricular ejection fraction |
| NT-proBNP | N-terminal pro-B-type natriuretic peptide |
| NYHA | New York Heart Association |
| SBP | systolic blood pressure |
| bpm | beats per minute |
| DBP | diastolic blood pressure |
| N.A. | not applicable |
| SD | standard deviation |
| BB | beta-blocker |
| CRT | cardiac resynchronization therapy |
| ICD | implantable cardioverter defibrillator |
| MRA | mineralocorticoid receptor antagonist |
| AE | adverse event |
| RRR | relative risk ratio |
| sac/val | LCZ696 |
| (in certain Figures) | |
| SAE | serious adverse event |
| IV | intravenous |
| AST | aspartate transaminase |
| ALT | alanine transaminase |
| INR | international normalised ratio |
| IQR | interquartile range |
| ICF | informed consent form |
| CI | confidence interval |
| HR | hazard ratio |
| LVAD | left ventricular assist device |
| NA | not available |
| RRR | relative risk ratio |

Study Drug LCZ696:

LCZ696 refers to the supramolecular complex trisodium [3((1S,3R)-1-biphenyl-4-ylmethyl-3-ethoxycarbonyl-1-butylcarbamoyl) propionate-(S)-3′-methyl-2′-(pentanoyl{2″-(tetrazol-5-ylate)biphenyl-4′-ylmethyl}amino)butyrate]hemipentahydrate. This compound and pharmaceutical compositions thereof have been previously disclosed in WO2007056546 and WO2009061713, whose preparative teachings are incorporated herein by reference.

### Example 1 TRANSITION Study

Study Design

TRANSITION (NCT02661217) is a randomized, parallel, open-label study comparing pre- and post-discharge (1-14 days) initiation of LCZ696 in patients with HFrEF, New York Heart Association (NYHA) class II-IV, left-ventricular ejection fraction (LVEF)≤40% following hemodynamic stabilization after an episode of ADHF, including patients with newly diagnosed HF (Pascual Figal D, et al. ESC Heart Fail 2018; 5(2):327-368).

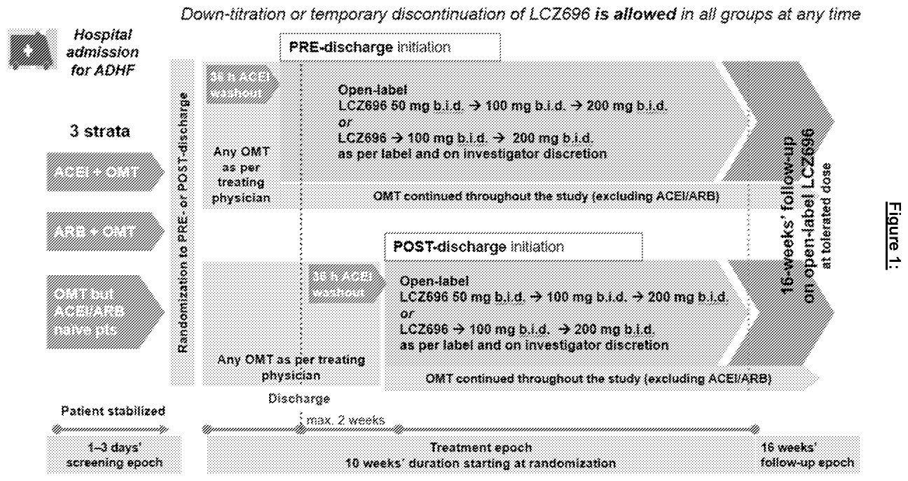

The study design comprises three Epochs (phases): the Screening Epoch; the Treatment Epoch defined as 10 weeks after Randomization; and 16-week Follow-up Epoch. Patients were stratified into three groups based on their pre-admission treatment status: (a) receiving an angiotensin-converting enzyme inhibitor (ACEI), (b) receiving an angiotensin receptor blocker (ARB), or (c) ACEI/ARB-naïve patients. Patients were randomized 1:1 within each stratum for initiation of LCZ696 treatment either pre- or post-discharge (see FIG. 1).

Study Population

The study population consists of patients hospitalized for an episode of acute decompensation of heart failure who are diagnosed with CHF NYHA class II-to-IV and reduced ejection fraction (LVEF 40%). Patients can be either with first presentation (de novo), or acute decompensation of HF due to deterioration in patients with a prior history of (chronic) HF.

Inclusion Criteria:
1. Patients hospitalized due to acute decompensated HF episode (ADHF) as primary diagnosis) and consistent Signs and Symptoms.
2. Diagnosis of HF New York Heart Association class II-to-IV and reduced ejection fraction: Left ventricular ejection fraction 40% at Screening.
3. Patients did not receive any IV vasodilators (except nitrates), and/or any IV inotropic therapy from the time of presentation for ADHF to Randomization.
4. Stabilized (while in the hospital) for at least 24 h leading to Randomization defined as:
   No need for IV diuretics in the past 24 h prior to signing ICF
   Systolic blood pressure (SBP) 110 mm Hg for at least 6 h prior to Randomization
5. Meeting one of the following criteria:
   Patients on any dose of ACEI or ARB at screening
   ACEI/ARB naïve patients and patients not on ACEI or ARB for at least 4 weeks before screening.

Key Exclusion Criteria:
1. History of hypersensitivity to the sacubitril, valsartan, or any ARBs, NEP inhibitors or to any of the LCZ696 excipients.
2. Symptomatic hypotension and/or a SBP<110 mm Hg or SBP>180 mm Hg prior to randomization.
3. End stage renal disease at Screening; or estimated GFR<30 mL/min/1.73 m2 as measured by the simplified Modification of Diet in Renal Disease (MDRD) formula at Randomization.
4. Serum potassium >5.4 mmol/L at Randomization.
5. Current hospitalization where patient does not receive treatment for decompensated HF.
6. Known history of hereditary or idiopathic angioedema or angioedema related to previous ACE inhibitor or ARB therapy.
7. Severe hepatic impairment, biliary cirrhosis and cholestasis.

The key eligibility criteria for the patients enrolled in the TRANSITION study compared to the PARADIGM-HF and TITRATION studies are shown in Table 1.

TABLE 1
Key eligibility criteria for the patients enrolled in the TRANSITION,
PARADIGM-HF, and TITRATION studies

| Variable | TRANSITION | PARADIGM-HF | TITRATION |
|---|---|---|---|
| Age | ≥18 years | | |
| NYHA class | II-IV | | |
| LVEF | ≤40% | ≤40%^{a} | ≤35% |
| Plasma BNP or | No pre-defined entry | (BNP ≥150 pg/mL or | No pre-defined |
| NT-proBNP | levels | NT-proBNP ≥600 | entry levels |
| levels | | pg/mL) or | |
| | | (BNP ≥100 pg/mL or | |
| | | NT-proBNP ≥400 | |
| | | pg/mL and | |
| | | hospitalization for HF | |
| | | within last 12 months) | |
| SBP | ≥110 mmHg | ≥100 mmHg | ≥100 mmHg |
| eGFR | ≥30 mL/min/1.73 m² | | |
| Clinical status | Inpatients | Outpatients | Inpatients and |
| | | | outpatients |
| Previous ACEI/ | Variable doses of | Stable dose of an ACEI/ | Variable doses of ACEI/ARB |
| ARB dose | ACEI/ARB or | ARB equivalent to enalapril | or treatment naïve^{b,c} |
| | treatment naïve^{b} | 10 mg/day for at least 4 weeks | |
| | | before the screening | |

^{a}LVEF eligibility criteria was initially ≤40% and changed in a protocol amendment to ≤35%
^{b}ACEI/ARB naïve defined as patients without any previous ACEI/ARB for ≥4 weeks before hospital admission
^{c}For outpatients, ACEI/ARB dose must have been stable for at least 2 weeks

Demographics and Baseline Characteristics and Baseline Treatment

Key baseline characteristics and medical history for the TRANSITION study, compared to the PARADIGM-HF, and TITRATION studies are presented in Table 2.

TABLE 2
Baseline characteristics and medical history

| | | | | p-values | |
|---|---|---|---|---|---|
| | | | | TRANSITION | |
| | | PARADIGM- | | vs. | TRANSITION |
| Baseline | TRANSITION | HF | TITRATION | PARADIGM- | vs. |
| parameters | (N = 993)* | (N = 8442) | (N = 498) | HF | TITRATION |
| Age (years), | 67 (11.9) | 64 (11.4) | 64 (11.4) | <0.0001 | <0.0001 |
| mean (SD) | | | | | |
| Male, % | 75 | 78 | 79 | 0.0264 | 0.1143 |
| Black patients, % | 1 | 5 | 5 | <0.0001 | 0.0002 |
| LVEF (%), | 29 (7.5) | 29 (6.2) | 30 (5.3) | 0.0042 | 0.0265 |
| mean (SD) | | | | | |
| NYHA class | <1/64/34/1 | 5/70/24/1 | 0/71/29/<1 | <0.0001^{†} | 0.0011^{†} |
| I/II/III/IV, % | | | | | |
| SBP (mmHg), | 124 (13.9) | 121 (15.3) | 131 (16.3) | <0.0001 | <0.0001 |
| mean (SD) | | | | | |
| DBP (mmHg), | 74 (10.75) | 74 (10.3) | 77 (9.6) | 0.0187 | <0.0001 |
| mean (SD) | | | | | |
| Heart rate (bpm), | 74 (12.9) | 72 (14) | 72 (11) | 0.0019 | <0.0001 |
| mean (SD) | | | | | |
| eGFR | 62 (19.9) | 68 (20.1) | 70 (23.5) | <0.0001 | <0.0001 |
| (mL/min/1.73 | | | | | |
| m²), mean (SD) | | | | | |
| <60 mL/min/ | 51 | 37 | 34 | <0.0001 | <0.0001 |
| 1.73 m², % | | | | | |
| Ischemic | 46 | 60 | 61 | <0.0001 | <0.0001 |
| etiology, % | | | | | |

Medical history

| | | | | | |
|---|---|---|---|---|---|
| First onset (de | 29 | 0 | 0 | N.A. | N.A. |
| novo) HF, % | | | | | |
| Prior | 49 | 63 | 56 | <0.0001 | 0.0156 |
| hospitalization | | | | | |
| for HF, % | | | | | |
| Hypertension, % | 75 | 71 | 40 | 0.0030 | <0.0001 |
| Diabetes, % | 46 | 34 | 12 | <0.0001 | <0.0001 |
| Atrial | | | | | |
| fibrillation, % | 48 | 37 | 27 | <0.0001 | <0.0001 |

^{†}p-value for NYHA classes is based on classes II, III and IV
*TRANSITION Full Analysis Set (N = 993)

At baseline, mean age was 67 years and 75% of patients were male. Mean LVEF was 29%, and 64% and 34% of patients were in NYHA Class II and III, respectively. In total, 286 (29%) patients had new-onset (de novo) HFrEF and 242 (24%) patients were ACEI/ARB naïve. Additional baseline characteristics of the TRANSITION study will be presented as poster (P6531) at the ESC congress in Munich on 28 Aug. 2018

Compared with the PARADIGM-HF and TITRATION studies, patients enrolled in the TRANSITION study were older and more likely to be female, in NYHA class III, with lower eGFR, and to have hypertension, atrial fibrillation and diabetes.

Fewer patients enrolled in the TRANSITION study had HF of known ischemic etiology compared with the PARADIGM-HF and TITRATION studies.

The comparison of the baseline characteristics of patients from the TRANSITION study with those from the PARADIGM-HF and TITRATION studies shows that TRANSITION recruited a more severe HFrEF population than the two previously conducted studies. The TRANSITION patient population was older with more severe HF symptoms, and with more comorbidities.

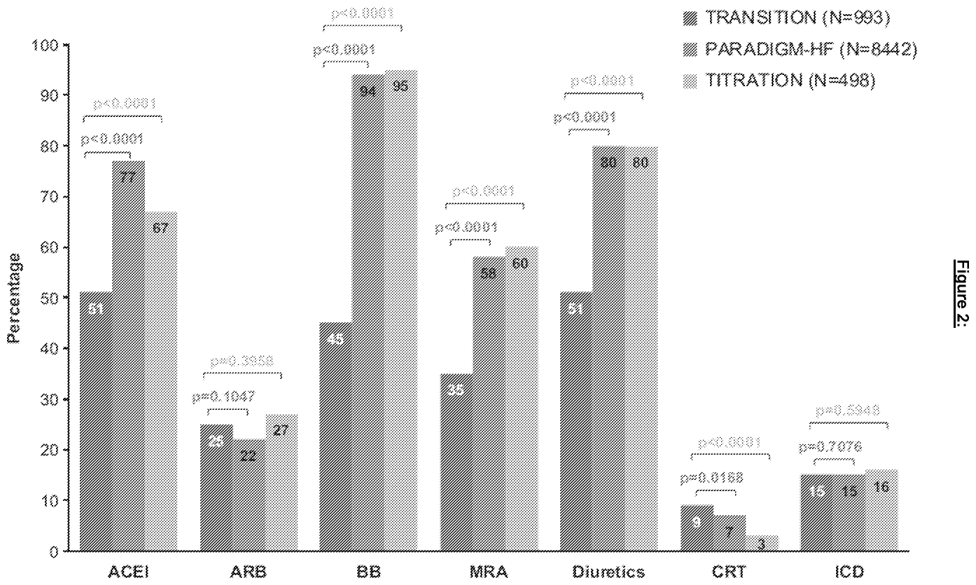

In the TRANSITION study, 242 (24%) patients were ACEI/ARB naïve, in contrast to PARADIGM-HF where all patients were on stable ACEI/ARB and TITRATION where 33 (6.6%) patients were ACEI/ARB naïve. The proportion of patients using beta-blockers, MRA, and diuretics prior to enrollment was lower in the TRANSITION study, compared with that in the PARADIGM-HF and TITRATION studies (see FIG. 2).

The TRANSITION study population represents a broad spectrum of HFrEF patients hospitalized due to an ADHF event who are stabilized and about to be discharged similar to the population in everyday clinical practice, including patients with new-onset (de novo) HFrEF and ACEI/ARB naïve patients.

Target Dose

The recommended starting dose of LCZ696 was 100 mg b.i.d.

A starting dose of 50 mg LCZ696 b.i.d. was considered for:

(1) patients not currently taking an angiotensin-converting enzyme (ACE) inhibitor or an angiotensin II receptor blocker (ARB);

(2) patients previously taking low doses of these agents;

(3) patients with moderate hepatic impairment (Child-Pugh grade B classification), or with AST/ALT values more than twice the upper limit of the normal range;

(4) patients with moderate renal impairment (eGFR 30-60 ml/min/1.73 m²).

The dose of LCZ696 was doubled every 2-4 weeks to achieve the target dose of 200 mg twice daily, as tolerated by the patient.

The basis for up-titration toward the target dose was taking into consideration tolerability (i.e., incidence of hypotension, renal or hepatic impairment and hyperkalemia) based on clinical evaluation and laboratory assessment. In case of patients experiencing tolerability issues, (a) adjusting concomitant medications, or (b) temporary down-titration, or (c) temporary or permanent discontinuation of LCZ696 was considered.

Primary Endpoint

The primary endpoint is the proportion of patients achieving the target dose of 200 mg LCZ696 b.i.d. at 10 weeks' post-randomization.

Secondary Endpoints

Secondary endpoints assess the tolerability of different doses of LCZ696 (200 mg b.i.d., 100 mg b.i.d., any dose) maintained for at least 2 weeks leading to week 10 after randomization, as well as the incidence of permanent LCZ696 discontinuation due to adverse events (AEs)

### REFERENCE

The study design and procedures can be found under www.clinicaltrials.gov, study number NCT02661217, which is herewith incorporated by reference.

Results

Study Population

Between February 2016 and December 2017, in total 1124 patients were screened and 1002 patients were randomised. Thirteen patients discontinued prior to randomisation, one of whom died during the screening period. A total of 111 patients did not meet the screening criteria. Thus, 500 patients were randomised to pre-discharge initiation and 502 to post-discharge initiation. Of these, 493 (99%) patients in the pre-discharge group and 489 (97%) patients in the post-discharge group received study medication.

The median time from admission to first dose of study drug was 7 days in the pre-discharge group and 10 days in the post-discharge group. Median time from randomisation to the first dose was 0 days [interquartile range (IQR) 0-1 days] and 3 days

(IQR 2-6 days) in pre-discharge and post-discharge groups, respectively. The median time from discharge to the first dose was −1 day in the pre-discharge group (IQR −2 to −1 days) and 1 day in the post-discharge group (IQR 1-4 days).

Baseline characteristics (Table 2 above) show that two-thirds of patients (64%) were in NYHA class II and 34% in NYHA class III at randomisation, as patients were expected to be stabilised after the acute event. Twenty-nine percent of patients were newly diagnosed (de novo) HF (n=286), and 24% (n=241) of the patients were ACEI/ARB-naive prior to the ADHF event (as per strata assignment), and 49% (n=485) had a prior hospitalisation for HF.

Initial Starting Dose of Sacubitril/Valsartan

A lower 24/26 mg bid starting dose was chosen by the investigators in 436 (88.4%) patients in the pre-discharge, and in 413 (84.5%) patients in the post-discharge group. The higher starting dose of 49/51 mg bid was used in the remaining patients.

Primary and Secondary Endpoints

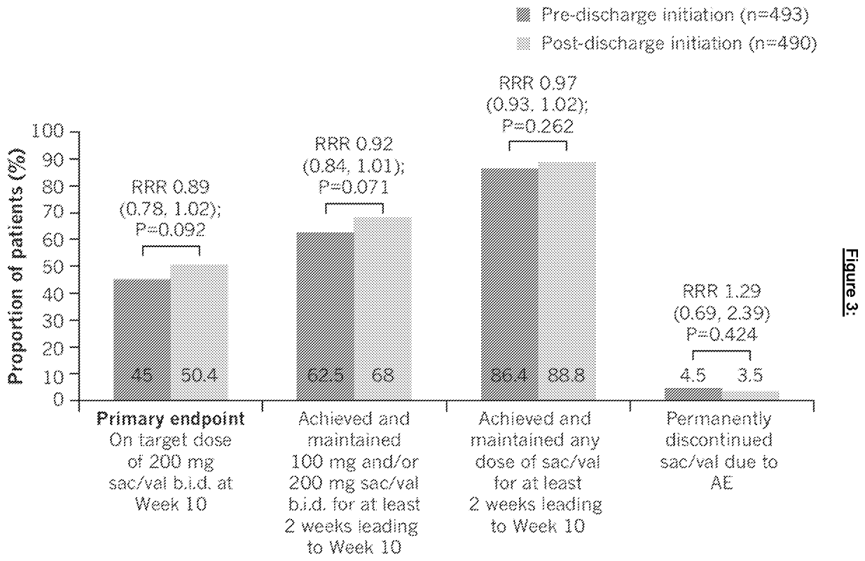

The proportion of patients who met the primary and secondary endpoints was comparable in the pre- and post-discharge initiation groups (see FIG. 3).

At 10 weeks after randomization, 45.0% of patients in the pre-discharge arm and 50.4% of patients in the post-discharge arm achieved the target dose of 200 mg twice daily (b.i.d.) LCZ696 (relative risk ratio [RRR] 0.893; 95% CI 0.783-1.019; p=0.092).

About two-thirds of patients initiated on LCZ696 pre-discharge (62.5%) and post-discharge (68%), achieved and maintained either 100 mg b.i.d or 200 mg b.i.d. for at least 2 weeks leading to week 10 after randomization (RRR 0.919; 95% CI 0.839-1.008; p=0.071). More than 86% of patients achieved and maintained any dose of LCZ696 for at least 2 weeks leading to week 10 after randomization in both groups (86.4% with pre-discharge initiation and 88.8% with post-discharge initiation) (RRR 0.973; 95% CI 0.929-1.020; p=0.262).

Rates of permanent discontinuation of study drug due to AEs were low: 4.5% among patients initiated pre-discharge and 3.5% among those initiated post-discharge (RRR 1.287; 95% CI 0.692-2.395; p=0.424).

There were no significant differences in the proportion of patients who met the primary endpoint between the ACEI or ARB strata, compared to those who were naive (relative RR 1.01; 95% CI 0.88-1.16). The analysis of the secondary endpoints confirmed a comparable tolerability of sacubitril/valsartan in both strata.

Predictors of Up-Titration Success

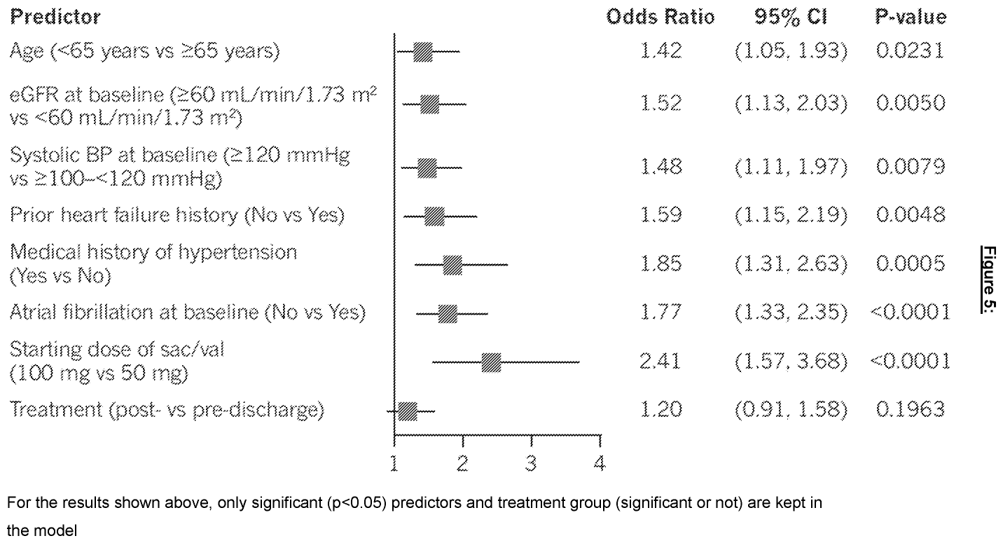

A multivariate logistic regression model was developed to identify baseline predictors of successful up-titration to LCZ696 200 mg b.i.d. target dose at the end of 10-week treatment. Odd ratios along with the 95% confidence intervals were constructed to identify those with high likelihood of achieving the target dose (see FIG. 5). In the analysis, significant (P<0.05) predictors of target-dose attainment within 10 weeks were age <65 years, SBP≥120 mmHg at baseline, history of hypertension, de novo HF, no atrial fibrillation at baseline, estimated glomerular filtration rate ≥60 mL/min/1.73 m2 at randomisation, and a sacubitril/valsartan starting dose of 49/51 mg bid. Assignment to pre- or post-discharge initiation of sacubitril/valsartan was not significant (OR 1.21; 95% CI 0.93-1.59), nor was prior use of an ACEI or ARB a significant predictor of up-titration success (OR 1.04; 95% CI 0.75-1.45) (FIG. 5).

Safety

Rates of AEs, serious AEs (SAEs), temporary and permanent treatment discontinuations during the 10-week period were comparable in the pre- and post-discharge initiation groups (see Table 3).

TABLE 3
Adverse events and treatment interruptions during the
10-week treatment period

| | Pre- | Post- | |
|---|---|---|---|
| | discharge | discharge | |
| | N = 497 | N = 496 | P- |
| | n(%) | n(%) | value* |
| Patients with ≥1 | 338 (68.0) | 323 (65.1) | 0.3349 |
| AE, n (%) | | | |
| Patients with ≥ | 94 (18.9) | 88 (17.7) | 0.6818 |
| SAE, n (%) | | | |
| Deaths, n (%) | 13 (2.6) | 10 (2.0) | 0.6740 |

Temporary treatment interruption, n (%)

| | | | |
|---|---|---|---|
| Due to AEs | 70 (14.1) | 55 (11.1) | 0.1803 |
| Due to SAEs | 21 (4.2) | 18 (3.6) | 0.7443 |
| Due to non- | 54 (10.9) | 42 (8.5) | 0.2374 |
| SAEs | | | |

Permanent treatment discontinuation, n (%)

| | | | |
|---|---|---|---|
| Due to AEs | 22 (4.4) | 20 (4.0) | 0.8749 |
| Due to SAEs | 15 (3.0) | 13 (2.6) | 0.8484 |
| Due to non- | 8 (1.6) | 7 (1.4) | 1.0000 |
| SAEs | | | |

*Fisher's Exact Test, Full analysis set

The most frequently reported AEs in patients in the pre-versus post-discharge group were hyperkalemia (11.1% vs 11.3%), hypotension (12.3% vs 9.1%), cardiac failure (6.8% vs 8.5%) and dizziness (5.6% vs 4.2%), respectively (see Table 4)

TABLE 4
Most common adverse events (≥4 of
patients in any group), during the 10-week
treatment epoch regardless of study drug relationship

| | Pre-discharge | Post-discharge | |
|---|---|---|---|
| | N = 497 | N = 496 | |
| Preferred term | n(%) | n(%) | P-value* |
| Hyperkalemia | 55 (11.1) | 56 (11.3) | 0.9201 |
| Hypotension | 61(12.3) | 45 (9.1) | 0.1229 |
| Cardiac failure | 34 (6.8) | 42 (8.5) | 0.3426 |
| Dizziness | 28 (5.6) | 21 (4.2) | 0.3795 |
| Peripheral edema | 17 (3.4) | 24 (4.8) | 0.2696 |
| Renal impairment | 25 (5.0) | 15 (3.0) | 0.1455 |
| Diarrhea | 12 (2.4) | 23 (4.6) | 0.0604 |
| Urinary tract infection | 20 (4.0) | 15 (3.0) | 0.4918 |

*Fisher's Exact Test, Full Analysis Set

SAEs were reported in 18.9% versus 17.7% of patients in the pre-versus post-discharge group, respectively (Table 3).

Of the reported SAEs, cardiac failure was the most common (7.0% vs 7.7% of patients in the pre-versus post-discharge group, respectively) (Table 5).

Similar frequency of acute kidney injury SAEs (1.2% vs 1.4% of patients in the pre-versus post-discharge group, respectively) (Table 5).

Hypotension and hyperkalemia SAEs were reported in <1% of patients in both treatment groups (Table 5).

Mortality rates were low in both treatment arms. Thirteen patients (2.6%) died in the pre-discharge arm and ten patients (2.0%) in the post-discharge arm (p=0.6740) (Table 3). None of the deaths were attributed to the study treatment by the investigator.

TABLE 5
Most common serious adverse events 0.5% in any group

| | Pre- | Post- | |
|---|---|---|---|
| | discharge | discharge | |
| | N = 497 | N = 496 | P- |
| Preferred term | n(%) | n(%) | value* |
| Number of patients with | 94 (18.9) | 88 (17.7) | 0.6818 |
| at least one SAE | | | |
| Hyperkalemia | 3 (0.6) | 2 (0.4) | 1.0000 |
| Hypotension | 4 (0.8) | 2 (0.4) | 0.6866 |
| Atrial fibrillation | 3 (0.6) | 4 (0.8) | 0.7255 |
| Cardiac failure(acute/chronic) | 35 (7.0) | 38 (7.7) | 0.7172 |
| Ventricular tachycardia | 3 (0.6) | 0 (0.0) | 0.2492 |
| Non-cardiac chest pain | 0 (0.0) | 3 (0.6) | 0.1242 |
| Pneumonia | 4 (0.8) | 3 (0.6) | 1.0000 |
| Respiratory tract infection | 0 (0.0) | 3 (0.6) | 0.1242 |
| Acute kidney injury | 6(1.2) | 7 (1.4) | 0.7890 |
| Renal failure | 3 (0.6) | 1 (0.2) | 0.6242 |
| Pulmonary edema | 3 (0.6) | 2 (0.4) | 1.0000 |
| Chronic obstructive pulmonary | 0 (0.0) | 4 (0.8) | 0.0619 |
| disease | | | |

*Fisher's Exact Test, Full Analysis Set

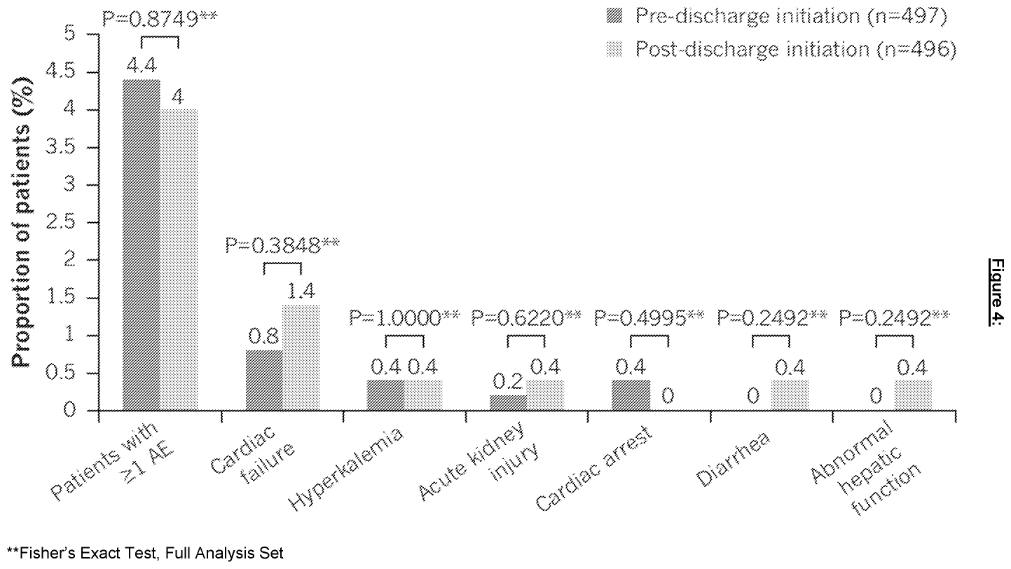

The most common AEs leading to permanent treatment discontinuations were cardiac failure and hyperkalemia (see FIG. 4)

Subgroup Analysis
A. Patients who are newly diagnosed with heart failure with a reduced ejection fraction (de novo HFrEF).

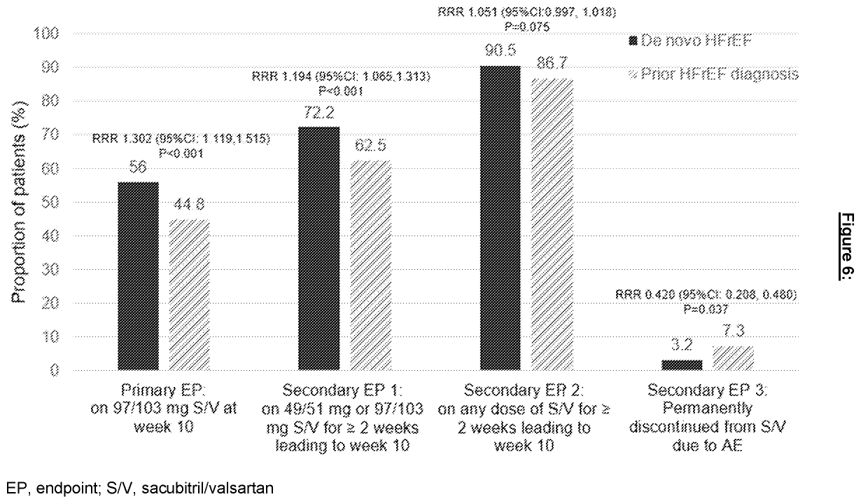

In TRANSITION (NCT02661217), 286 patients (28.8%) were de novo HFrEF and 705 (71.0%) had a previous diagnosis of HFrEF. At baseline, de novo HF patients were younger, had lower systolic blood pressure, higher pulse rate, more frequently non-ischemic HF, lower serum creatinine, higher e-GFR, lower high-sensitive Troponin T levels, and lower presence of common HF comorbidities. Significantly more de novo HF patients achieved the target dose compared to subjects with a prior HF diagnosis (56.0% vs. 44.8%, RRR 1.30, 95% CI 1.12, 1.52, P<0.001) with 90% able to achieve and maintained any sacubitril-valsartan dose at week 10 (FIG. 6). Overall incidence of AEs was comparable between groups a serious AEs, temporary and permanent discontinuation of sacubitril-valsartan due to AEs were lower in patients with de novo HFrEF (Table 6).

TABLE 6
Overall incidence of AEs in de novo patients

| | | Previous | |
|---|---|---|---|
| | De novo | diagnosis | |
| | HFrEF | of HFrEF | |
| | (N = 286) | (N = 705) | |
| Event | n (%) | n(%) | P-value |
| At least one AE | 178 (62.2) | 478 (67.8) | 0.103 |
| Selected AEs of interest | | | |
| Hyperkalemia | 24 (8.4) | 85 (12.1) | 0.116 |
| Hypotension | 26 (9.1) | 108 (10.9) | 0.263 |
| Cardiac failure | 13 (4.5) | 58 (8.2) | 0.042 |
| Renal failure | 3 (1.0) | 16 (2.3) | 0.306 |
| Blood creatinine increased | 3 (1.0) | 26 (3.7) | 0.023 |
| Renal impairment | 8 (2.8) | 32 (4.5) | 0.284 |
| At least one serious AE | 33 (11.5) | 130 (18.4) | 0.008 |
| Death | 1 (0.3) | 5 (0.7) | 0.679 |
| Treatment interruption due to AE | 22 (7.7) | 87 (12.3) | 0.034 |

B. Patients who are naïve to angiotensin converting-enzyme inhibitor (ACEI) or angiotensin receptor blocker (ARB).

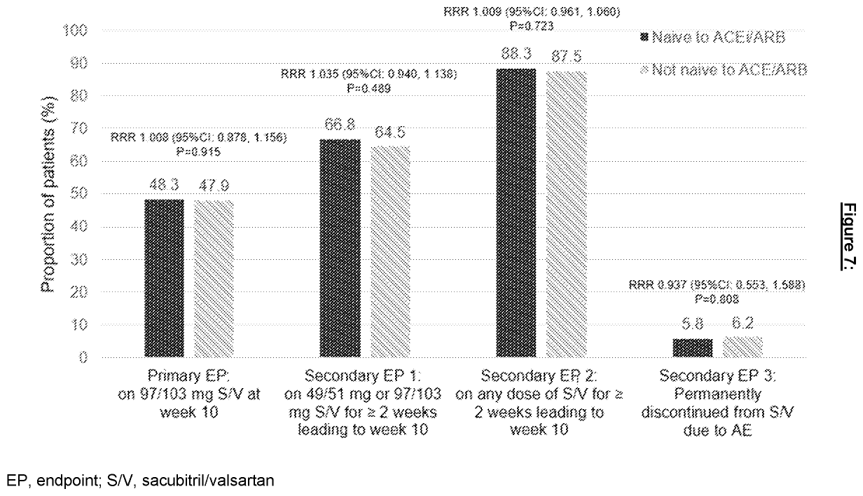

In TRANSITION, 326 patients (32.9%) were ACEI/ARB naïve and 665 (67.1%) were not. At baseline, ACEI/ARB naïve patients had lower systolic blood pressure, serum creatinine, and presence of common HF comorbidities but had higher pulse rate and more often non-ischemic HF etiology. Similar proportions of ACEI/ARB naïve patients achieved the target dose compared to subjects with prior ACEI/ARB use (48.3% vs. 47.9%, RRR 1.008, 95% CI 0.878, 1.156) with 88% of subjects in both groups maintained on any sacubitril-valsartan dose at week 10 (FIG. 7). Overall incidence of AEs, serious AEs and permanent discontinuation of sacubitril-valsartan due to AEs were comparable between groups (Table 7).

TABLE 7
Overall incidence of AEs in naive patients

| | Naive to | Not naive to | |
|---|---|---|---|
| | ACEI/ARB | ACEI/ARB | |
| | (N = 326) | (N = 665) | |
| Event | n(%) | n(%) | P-value |
| At least one AE | 210 (64.4) | 446 (67.1) | 0.432 |
| Selected AEs of interest | | | |
| Hyperkalemia | 20 (7.1) | 86 (12.9) | 0.005 |
| Hypotension | 29 (8.9) | 79 (11.9) | 0.193 |
| Cardiac failure | 17 (5.2) | 54 (8.1) | 0.115 |
| Renal failure | 6 (1.8) | 13 (2.0) | 1.000 |
| Blood creatinine increased | 6 (1.8) | 23 (3.5) | 0.228 |
| Renal impairment | 8 (2.5) | 32 (4.8) | 0.086 |
| At least one serious AE | 47 (14.4) | 116 (17.4) | 0.237 |
| Treatment interruption | 32 (9.8) | 77 (11.8) | 0.450 |
| due to AE | | | |

Conclusion:

In TRANSITION, about half of the HFrEF patients stabilized after an acute HF decompensation event achieved the recommended target dose of 200 mg LCZ696 b.i.d. within 10 weeks

The incidence of adverse events and discontinuations of LCZ696 due to adverse events was similar in pre-versus post-discharge groups

Patients with fewer comorbidities, higher systolic blood pressure or newly diagnosed HF were more likely to tolerate the up-titration of LCZ696 to target dose within 10 weeks

Initiation of LCZ696 in a wide range of HFrEF patients, in-hospital or shortly after discharge, was feasible and overall well tolerated.

Patients with de novo HFrEF can be safely initiated on sacubitril-valsartan and are more likely to achieve the target dose and maintain S/V treatment by week 10, compared to patients with a previous diagnosis of HFrEF.

Patients with HFrEF, stabilized after an ADHF event, who are naïve to ACEI/ARB can be safely initiated, up-titrated and maintained on S/V, similar to patients previously treated with ACEI/ARB.

### Example 2. PIONEER-HF Study

Study Design

PIONEER-HF (NCT02554890) is a prospective, multicenter, double-blind, randomized, active-controlled trial, designed to assess the efficacy, safety and tolerability of LCZ696 in patients hospitalized for acute HFrEF.

Consenting adults ≥18 years of age were included with an EF≤40% who had either an N-terminal-pro b-type natriuretic peptide (NT-proBNP)≥1600 pg/mL or BNP≥400 pg/mL. The study rational and design is described in by Velazquez E J, et al. .in Am. Heart J 2018; 198: 145-51, which is herewith incorporated by reference.

Study Population

Patients were eligible for participation no earlier than 24 hours and up to 10 days from initial presentation and were randomly assigned 1:1 to in-hospital initiation of LCZ696 titrated up to 97/103 mg twice daily vs. enalapril titrated to 10 mg twice daily for 8 weeks. The starting dose and all subsequent doses were selected to optimize the highest tolerated dose based on a systolic blood pressure (SBP) algorithm.

Inclusion Criteria:
Patients ≥18 y of age with the capacity to provide written informed consent.
Currently hospitalized for a primary diagnosis of HF, including symptoms and signs of fluid overload.
Eligible patients will be randomized no earlier than 24 hours and up to ten days after presentation while still hospitalized as long as meet the following definition of stable status:
   SBP≥100 mm Hg for the preceding 6 hours prior to randomization; no symptomatic hypotension
   No increase (intensification) in i.v. diuretic dose within last 6 hours prior to randomization
   No i.v. inotropic drugs for 24 hours prior to randomization
   No i.v. vasodilators including nitrates within last 6 hours prior to randomization
Left ventricular EF 40% within the past 6 m (including current hospitalization) using echocardiography, multi gated acquisition scan (MUGA), CT scanning, MRI or ventricular angiography, provided no subsequent study documented an EF of >40%
Elevated NT-proBNP≥1600 pg/mL OR BNP≥400 pg/mL during current hospitalization.

Key Exclusion Criteria:
Currently taking LCZ696 tablets or any use within the past 30 days.
Enrollment in any other clinical trial involving an investigational agent or investigational device.
History of hypersensitivity, known or suspected contraindications, or intolerance to any of the study drugs, including ACEIs, ARBs, or Sacubitril (NEP inhibitor).
Patients with a known history of angioedema related to previous ACE inhibitor or ARB therapy.
Requirement of treatment with both ACE inhibitor and ARB.
eGFR<30 ml/min/1.73 m2 as measured by the simplified Modification of Diet in Renal Disease (MDRD) formula at screening.
Serum potassium >5.2 mEq/L at screening.
Known hepatic impairment (as evidenced by total bilirubin >3 mg/dL, or increased ammonia levels, if performed), or history of cirrhosis with evidence of portal hypertension such as varices
Acute coronary syndrome, stroke, transient ischemic attack; cardiac, carotid or other major CV surgery; percutaneous coronary intervention (PCI) or carotid angioplasty, within one month prior to Visit 1.
Pregnant or nursing (lactating) women, where pregnancy is defined as the state of a female after conception and until the termination of gestation, confirmed by a positive hCG laboratory test.
Women of child-bearing potential, defined as all women physiologically capable of becoming pregnant, including women whose career, lifestyle, or sexual orientation precludes intercourse with a male partner and women whose partners have been sterilized by vasectomy or other means, UNLESS they are using two birth control methods

Demographics and Baseline Characteristics and Baseline Treatment

At 129 US sites, 887 patients (72% male; 36% African-American) with a mean (±SD) age of 61 (±14) years were randomized at a median of 4 (IQR 2) days from presentation. Among patients randomized, 303 (34%) reported no prior history of HF (de novo) and 463 (52%) were angiotensin converting-enzyme inhibitor (ACEi)/angiotensin receptor blocker (ARB) naïve. The median (IQR) EF was 0.25 (0.11) and median (25th 75th) enrolling NT-proBNP was 4817(3105, 8745) pg/ml. At baseline, the median (IQR) SBP and serum creatinine were 118 (22) mmHg and 1.28 (0.45) mg/dl, respectively. By week 8, 511 (58%) patients had achieved the maximum possible dose.

Primary Endpoint
Percentage change from baseline in N-terminal pro-brain natriuretic peptide (NT-proBNP), [Time Frame: Baseline, Week 4 and Week 8]

The primary objective of this study is to assess the effect of in-hospital initiation of LCZ696 vs. enalapril on the time-averaged percentage change of NT-proBNP from baseline in patients who have been stabilized following hospitalization for ADHF and reduced ejection fraction (left ventricular ejection fraction [LVEF]≤40%) between week 4 and 8.

Secondary Endpoints
Number of patients with incidences of Symptomatic hypotension [Time Frame: 8 weeks]
   Examine the effect of LCZ696 vs. enalapril on incidence of symptomatic hypotension during 8 weeks of treatment
Number of patients with incidences of hyperkalemia [Time Frame: 8 weeks]
   Hyperkalemia is defined as Potassium level >5.5 meg/l
Number of incidences of Angioedema [Time Frame: 8 weeks]
   Angioedema is a type of abrupt swelling that occurs under the skin and/or mucous membranes and is often localized to the head, neck, throat, and/or tongue, but may occur elsewhere, including the genitalia and intestines.
Change from baseline in high sensitivity troponin (hs-Troponin) [Time Frame: Baseline, Week 4 and Week 8]
Change from baseline in urinary cGMP [Time Frame: Baseline, Week 4 and Week 8]
Change from baseline in BNP to NTproBNP ratio [Time Frame: baseline, Week 4 and Week 8]

### REFERENCE

The study design and procedures can be found under www.clinicaltrials.gov, study number NCT02554890, which is herewith incorporated by reference.

Results

Study Population

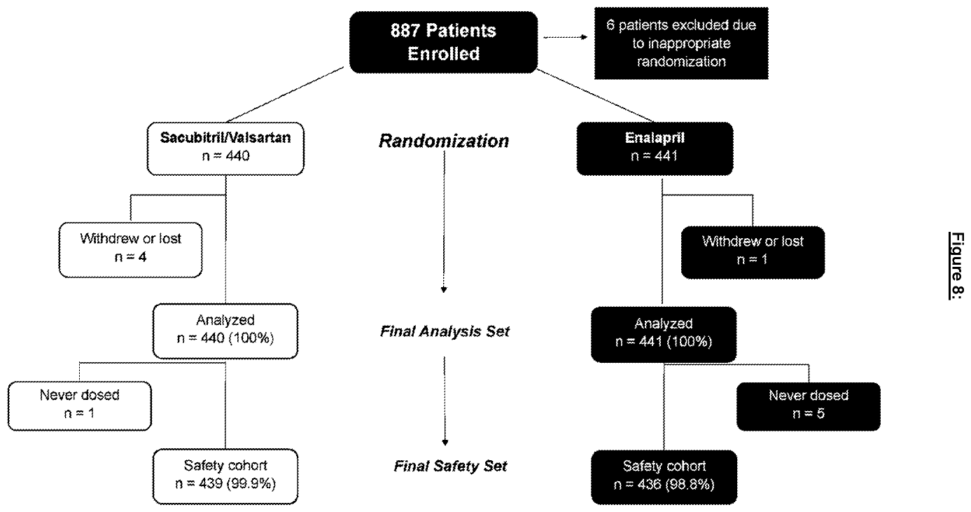

A number of 887 patients were enrolled at 129 participating centers in the United States. A total of 6 (0.7%) patients were inappropriately randomized and did not receive any study drug and were prospectively omitted from all analyses. The final analytical cohort included 881 patients, 440 randomly assigned to receive LCZ696 and 441 randomly randomized to receive enalapril (see FIG. 8).

Patients were enrolled a median (25th, 75th) of 68 (48, 98) hours from initial presentation. HF signs and symptoms were highly prevalent at the time of randomization with 61.7% having peripheral edema and 32.9% with lung rales. Baseline characteristics, including demographics, clinical findings, medication history, and index hospitalization details were similar between the 2 treatment groups. Patients mean age was 61±14 years, 635 (72%) were male, and 316 (36%) self-identified as black. The index hospitalization was the first diagnosis of HF for 303 (34%) patients. Among the 576 (65%) patients diagnosed with HF before the index hospitalization, 343 (60%) reported at least 1 prior hospitalization for HF within the past 12 months and 351 (61%) reported NYHA functional class III or IV symptoms within the past month. At the time of hospital admission, 459 (52%) patients were not receiving ACEi/ARB therapy.

At randomization, the median SBP was 118 (110, 132) mm Hg and 23% patients had a SBP<110 mm Hg. The baseline median LVEF was 0.25 (0.20, 0.30) and the median serum creatinine at baseline was 1.28 (1.06, 1.51) mg/dL. The screening local laboratory median NT-proBNP was 4812 (3050, 8745) pg/mL and median BNP was 1063 (718, 1743) pg/mL. At randomization, the majority of patients (85%) were treated with loop diuretic. During the index hospitalization and prior to randomization, 814 (93%) patients received intravenous furosemide therapy, 97 (11%) received care in an intensive care unit, and 68 (7.7%) required an intravenous inotrope. The median duration of the index hospitalization was 5.20 (4.09, 7.24) days.

Study Treatment and Follow Up

At least one dose of study drug was administered to 875 patients, 439 randomized to LCZ696 and 436 to enalapril, the pre-defined cohort for safety analyses. Excluding discontinuation due to death, study drug was prematurely discontinued prior to 8 weeks in 87 (19.6%) patients assigned to LCZ696 and 90 (20.3%) assigned to enalapril. Overall 5 patients, 4 randomized to LCZ696 and 1 to enalapril, were lost to follow-up or withdrew consent (n=1); their data were censored at a median of 37 days. By the week 8 study visit, the maximum target dose had been achieved in 245 (56%) patients in the LCZ696 and 270 (62%) in the enalapril arm.

Outcomes

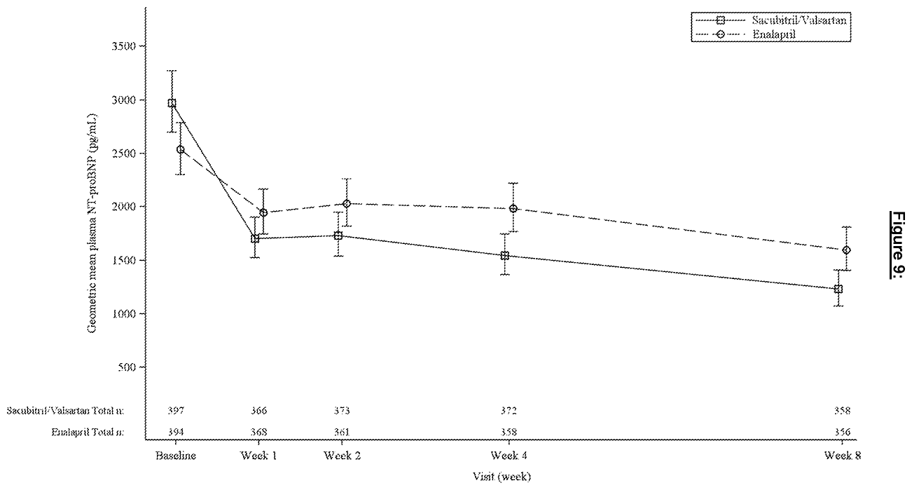

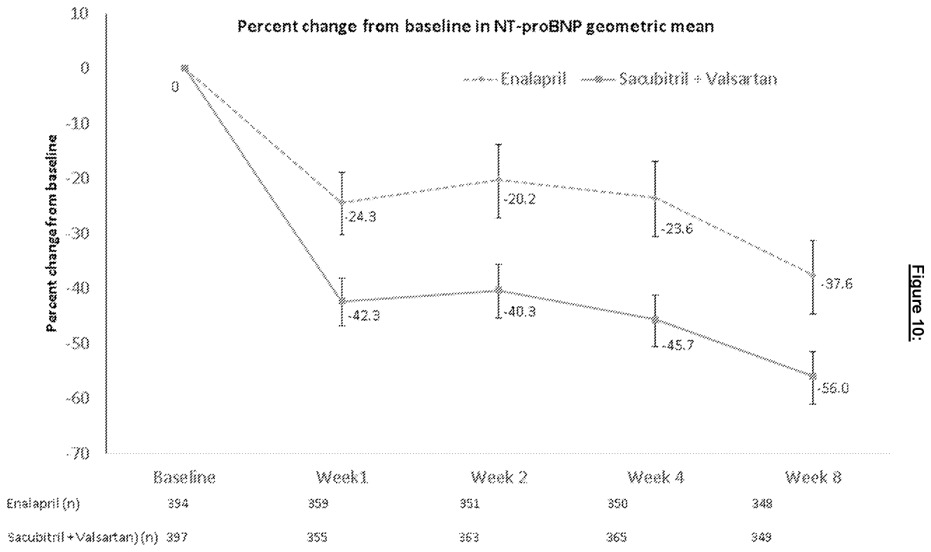

NT-proBNP levels declined in both treatment arms, with a significantly greater reduction in patients receiving LCZ696 compared with enalapril (LCZ696 vs. enalapril ratio of time-averaged change from baseline to the geometric mean of weeks 4 and 8: 0.71, 95% CI 0.63 to 0.81; p<0.001) (FIGS. 9 and 10, Table 8). The greater reduction in NT-proBNP concentration by LCZ696 treatment was apparent as early as week 1 (0.76, 95% CI 0.69 to 0.85; p<0.001) and at every subsequent visit.

TABLE 8
Outcomes

| | LCZ696 | Enalapril | HR (95% CI)^{*,†} | |
|---|---|---|---|---|
| Outcomes | (n = 440) | (n = 441) | (LCZ696 vs. Enalapril) | p-value |
| Composite^{‡} | 41 (9.3) | 74 (16.8) | 0.54 (0.37, 0.79) | <0.002 |
| Death | 10 (2.3) | 15 (3.4) | 0.66 (0.30, 1.48) | 0.311 |
| HF rehospitalization | 35 (8.0) | 61 (13.8) | 0.56 (0.37, 0.84) | <0.01 |
| LVAD implantation | 1 (0.2) | 1 (0.2) | 0.99 (0.06, 15.97) | 0.999 |
| Listing for cardiac transplant | 0 (0.0) | 0 (0.0) | NA | |
| Expanded composite^{§} | 262 (59.5) | 275 (62.4) | 0.409 | 0.429 |
| Unplanned outpatient visits requiring | 2 (0.5) | 2 (0.5) | 1.00 (0.14, 7.07) | 0.997 |
| intravenous diuretics | | | | |
| Additional HF drug | 75 (17.0) | 83 (18.8) | 0.89 (0.65, 1.22) | 0.472 |
| Increase in diuretic | 242 (55.0) | 239 (54.2) | 0.84 (0.84, 1.21) | 0.915 |
| dose >50% | | | | |

Data presented as number (%), unless otherwise indicated.
^{*}Hazard ratio is based on the Cox model, and p-value is based on the log-rank test.
^{†}All assessments are adjusted for baseline NT-proBNP.
^{‡}Composite included death, rehospitalization for HF, LVAD implantation, or listing for cardiac transplant.
^{§}Expanded composite included death, rehospitalization for HF, LVAD implantation, listing for cardiac transplant, unplanned outpatient visits requiring IV diuretics, or increase >50% in diuretic dose.

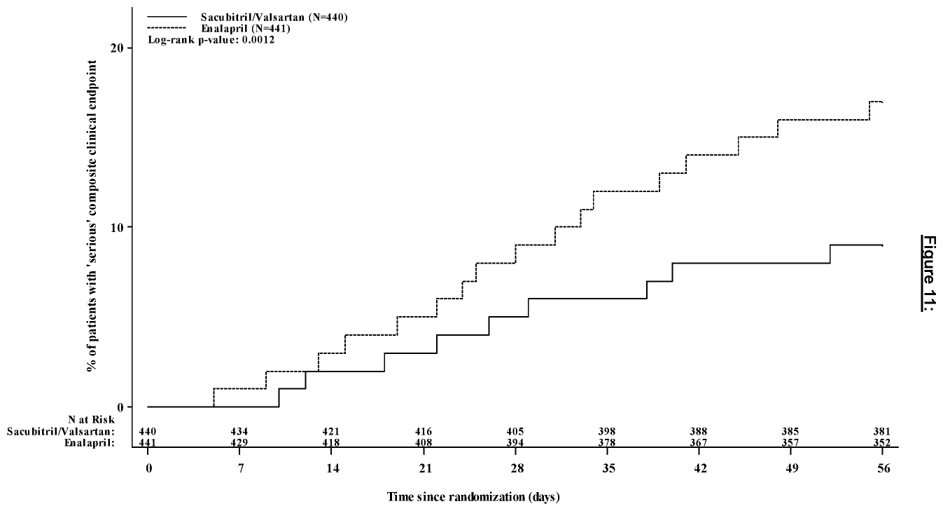

Clinical outcomes in the LCZ696 compared with enalapril groups are summarized in Table 8. For the clinically relevant composite endpoint of death, rehospitalization for HF, LVAD implantation, or listing for cardiac transplant, patients randomized to LCZ696 experienced a significantly lower risk compared with patients randomized to enalapril (n=41 vs. 74, HR 0.54, 95% CI 0.37 to 0.79; p=0.001) (FIG. 11).

Key safety endpoints were similar between the LCZ696 and enalapril groups in regard to worsening renal function (13.6% vs. 14.7%, HR 0.93, 95% CI 0.67 to 1.28), hyperkalemia (11.6% vs. 9.3%, HR 1.25, 95% CI 0.84 to 1.84) and symptomatic hypotension (10.0% vs. 9.1%, HR 1.10, 95% CI 0.73 to 1.66). Additionally, no clinically significant differences in serum creatinine, potassium, or SBP were observed between the two groups throughout the study period. Blinded adjudication, confirmed one angioedema event in the LCZ696 group (in a white patient) and six (all in patients who self-identified as black) in the enalapril group (0.2% vs. 1.4%, HR 0.17, 95% CI 0.02 to 1.38). There was no statistically significant between-group difference in rate of permanent study drug discontinuation due to any adverse event.

Subgroup Analysis

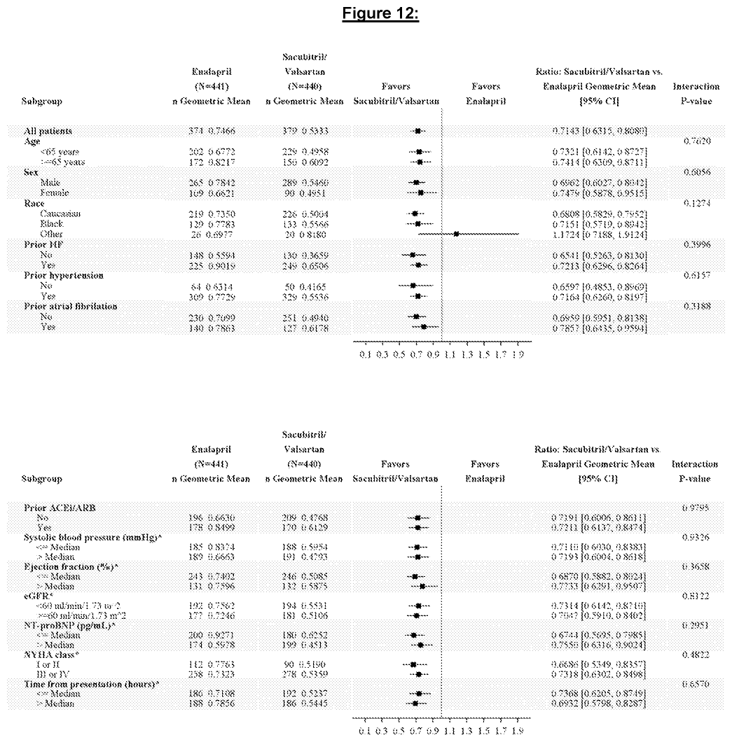

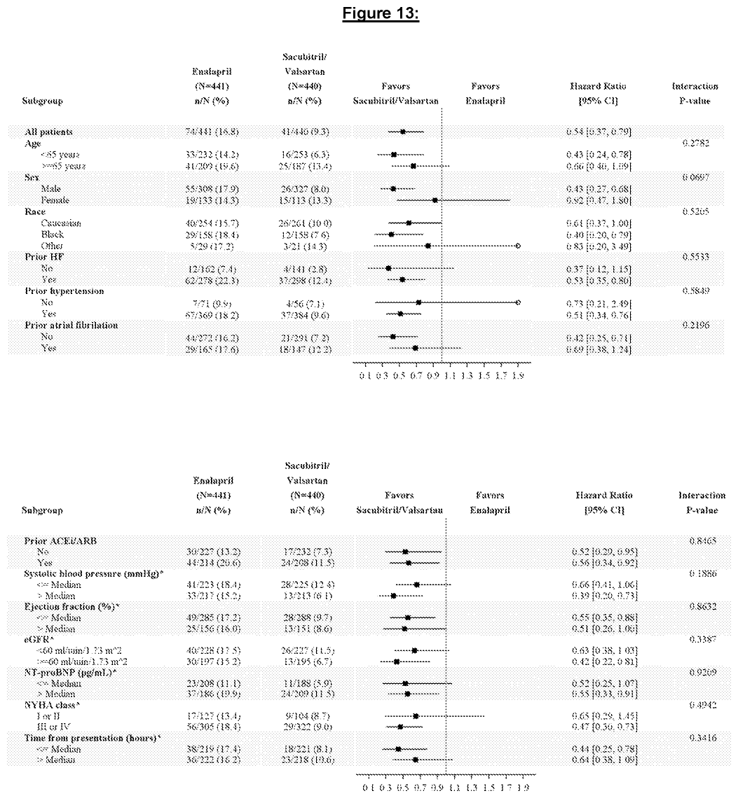

Subgroup analyses based on demographic and clinical characteristics of interest showed consistency of the effect of LCZ696 compared with enalapril on the primary outcome (FIG. 12) and the composite clinical endpoint, with no significant treatment interactions by subgroup identified (FIG. 13). Significant treatment interactions by subgroup for the key safety endpoints of worsening renal function, hyperkalemia, and symptomatic hypotension were also not found.

Cardiovascular Biomarker Analysis

Circulating high-sensitivity cardiac troponin (hsTn) and soluble ST2 (sST2) reflect myocardial stress in patients with heart failure (HF). Production of cyclic guanosine 3′5′ monophosphate (cGMP) in response to activation of natriuretic peptide receptors reduces cardiac afterload and preload.

Circulating hsTnT, sST2, and urinary cGMP at baseline, 1, 2 (sST2, cGMP), 4, and 8 weeks (n=694 with all baseline biomarkers) were measured. Ratios of geometric means (timepoint/baseline) were determined and compared as a ratio for sacubitril/valsartan vs. enalapril.

Compared with enalapril, sacubitril/valsartan led to a significantly greater decline in hsTnT and sST2. This effect emerged as early as 1 week for sST2 and was significant for both at 4 weeks with a 16% greater reduction in hsTnT (P<0.001) and 9% greater reduction in sST2 (P=0.0033). Serial urinary cGMP increased with sacubitril/valsartan compared with enalapril (P<0.001, 1 week). The significant differences between treatment groups for each biomarker were sustained at 8 weeks. In an exploratory multivariable-adjusted analysis of cardiovascular death or HF-rehospitalization, the concentrations of hsTnT, sST2 at week 1 were significantly associated with subsequent outcome.

### DISCUSSION

The PIONEER-HF trial was performed to evaluate the use of a neprilysin inhibitor added to a renin-angiotensin system inhibitor, as compared with a renin-angiotensin system inhibitor alone, in the treatment of patients who were hospitalized for acute heart failure. The initiation of sacubitril-valsartan therapy after hemodynamic stabilization led to a greater reduction in the NT-proBNP concentration than enalapril therapy, a difference that was evident by the first week.

The beneficial effect of sacubitril-valsartan on the concentration of NT-proBNP, which is a biomarker of neurohormonal activation, hemodynamic stress, and subsequent cardiovascular events, was accompanied by a reduction in the concentration of high-sensitivity cardiac troponin T, which is a biomarker of myocardial injury associated with abnormalities of cardiac structure and function and with a worse prognosis among patients with heart failure. The rates of renal dysfunction, hyperkalemia, and symptomatic hypotension did not differ significantly between the sacubitril-valsartan group and the enalapril group. Furthermore, in an analysis of exploratory clinical outcomes, the in-hospital initiation of sacubitril-valsartan therapy was associated with a lower rate of rehospitalization for heart failure at 8 weeks than enalapril therapy.

The results of the PIONEER-HF trial extend the evidence base regarding the use of sacubitril-valsartan to populations for which there had been limited or no data, including patients who are hospitalized for acute decompensated heart failure, patients who have new heart failure, patients who have not been exposed to high doses of guideline-directed medications for heart failure, and patients who are not receiving conventional renin-angiotensin system inhibitors. In addition, 35.9% of the patients in our trial identified as black, and there is limited evidence from previous clinical studies regarding the use of sacubitril-valsartan among black patients. The favorable effect of sacubitril-valsartan, as compared with enalapril, was evident from the in-hospital initiation of treatment and continued to be present during the transition to home and throughout the subsequent “vulnerable period” during which morbidity and mortality among patients with acute decompensated heart failure remain high.

The finding that the rates of renal dysfunction, hyperkalemia, and symptomatic hypotension did not differ significantly between the sacubitril-valsartan group and the enalapril group is reassuring, especially among patients with acute decompensated heart failure, who are at a high risk for hemodynamic instability. In addition, in the sacubitril-valsartan group, there was only one case of angioedema, with no cases among black patients. Results from previous trials of sacubitril-valsartan, most notably the PARADIGM-HF trial, were limited to ambulatory outpatients who had received established high doses of an ACE inhibitor or ARB, as well as the highest doses of enalapril and sacubitril-valsartan during sequential single-blind run-in periods before randomization.

The PIONEER-HF trial made use of the lowest starting dose of sacubitril-valsartan (24 mg of sacubitril with 26 mg of valsartan), with which there was less experience. The PIONEER-HF trial set specific requirements for the in-hospital initiation of sacubitril-valsartan therapy. Patients were required to have had a systolic blood pressure of at least 100 mm Hg for the preceding 6 hours, with no increase in the dose of intravenous diuretics and no use of intravenous vasodilators during the preceding 6 hours and no use of intravenous inotropes during the preceding 24 hours. Sacubitril-valsartan therapy was initiated at a low dose among patients with lower systolic blood pressure, and the dose was adjusted according to a prespecified algorithm. A washout period of 36 hours was used to ensure that patients who had previously been taking an ACE inhibitor or ARB did not have any overlapping medication effects. Despite these precautions, approximately 20% of the patients in each treatment group had discontinued treatment by 8 weeks, in most cases because of an adverse event.

Taken together, these considerations suggest that the initiation of any neurohormonal agent in this population should be performed cautiously. There are several limitations of our trial. The in-hospital initiation phase, which included the provision of placebo alone for the first two doses in the sacubitril-valsartan group and then mandatory observation for 6 hours after the third dose, may have prolonged the length of stay. These elements of the protocol were necessary to preserve blinding, maintain protocol consistency, and ensure patient safety. In addition, approximately 0.5% of the patients were lost to follow-up and 15% had missing data on the NT-proBNP concentration, although the results for the primary efficacy outcome remained significant in an analysis with multiple imputation.

### CONCLUSION

In conclusion, among patients who were hospitalized for acute decompensated heart failure, the initiation of sacubitril-valsartan therapy resulted in a significantly greater reduction in the NT-proBNP concentration than enalapril therapy. Compared with enalapril, sacubitril/valsartan reduces myocardial injury and haemodynamic stress as reflected by biomarkers (hsTnT, sST2, and urinary cGMP), with an onset that is apparent within 1-4 weeks.

There were no significant differences between the sacubitril-valsartan group and the enalapril group with regard to the rates of renal insufficiency, hyperkalemia, symptomatic hypotension, and angioedema.

1. A method of treating heart failure with reduced ejection fraction in a patient comprising administering to said patient in need thereof sacubitril and valsartan in a 1:1 molar ratio, wherein treatment is initiated shortly after an acute decompensation heart failure episode of said patient, wherein the term shortly refers to a time period starting with medical stabilization after the end of acute heart failure treatment and up to and including 10 days after an acute decompensation heart failure episode.

2. The method according to claim 1, wherein the treatment is initiated while the patient is still hospitalized due to the acute decompensation heart failure episode.

3. The method according to claim 1, wherein the patient is hemodynamically stable.

4. The method according to claim 3, wherein the hemodynamically stable patient is characterized by at least one of the following:
(i) a systolic blood pressure ≥100 mm Hg during 6 hours before initiation of treatment,
(ii) no increase in IV diuretics or use of IV vasodilators during 6 hours before initiation of treatment, or
(iii) no IV inotropes administered during 24 hours before initiation of treatment.

5. The method according to claim 3, wherein the hemodynamically stable patient is characterized by
(i) a systolic blood pressure ≥100 mm Hg during 6 hours before initiation of treatment,
(ii) no increase in IV diuretics or use of IV vasodilators during 6 hours before initiation of treatment, and
(iii) no IV inotropes administered during 24 hours before initiation of treatment.

6. (canceled)

7. The method according to claim 1, wherein the patient suffers from heart failure with reduced ejection fraction classified as NYHA class II, III or IV and wherein the patient has a reduced left ventricular ejection fraction (LVEF) of ≤40%.

8. (canceled)

9. The method according to claim 1, wherein the patient is a de novo patient not having been diagnosed as suffering from heart failure with reduced ejection fraction prior to said acute decompensation heart failure episode mentioned in claim 1.

10. (canceled)

11. The method according to claim 1, wherein the patient is an ACEI/ARB naïve patient not having received an ACEI or ARB or both prior to said acute decompensation heart failure episode mentioned in claim 1.

12. The method according to claim 1, wherein the patient achieves a target dose of 200 mg of sacubitril and valsartan in a 1:1 molar ratio b.i.d.

13-14. (canceled)

15. The method according to claim 12, wherein the target dose is reached after titration from a starting dose of 50 mg of sacubitril and valsartan in a 1:1 molar ratio b.i.d. increasing to the target dose during an up-titration period from about 2 to about 10 weeks.

16. The method according to claim 15, wherein the starting dose of 50 mg of sacubitril and valsartan in a 1:1 molar ratio b.i.d. is used in a patient
(i) not taking an ACEI or ARB before initiation of treatment;
(ii) with moderate hepatic impairment (Child-Pugh grade B classification), or with AST/ALT values more than twice the upper limit of the normal range before initiation of treatment; or
(iii) with moderate renal impairment (eGFR 30-60 ml/min/1.73 m2) before initiation of treatment.

17-21. (canceled)

22. The method according to claim 15, wherein the up-titration period is from about 2 to about 6 weeks.

23. (canceled)

24. The method according to claim 1, wherein sacubitril and valsartan in a 1:1 molar ratio refers to a combination of a 1:1 molar ratio of
(i) valsartan or a pharmaceutically acceptable salt thereof; and
(ii) sacubitril or a pharmaceutically acceptable salt thereof.

25. The method according to claim 24, wherein sacubitril and valsartan in a 1:1 molar ratio is provided in the form of the compound of the formula (I)
[(A₁)(A₂)](Na⁺)₃.xH₂O (I)
wherein
A₁ is valsartan in the dianionic form;
A₂ is sacubitril in the anionic form;
Na⁺ is a sodium ion; and
x is 0.5 to 3.5.

26. (canceled)

27. The method according to claim 25, wherein sacubitril and valsartan in a 1:1 molar ratio is provided in the form of the compound of the formula (I), wherein x is 2.5.

28. The method according to claim 27, wherein sacubitril and valsartan in a 1:1 molar ratio is provided in the form of the compound of the formula (I), which is trisodium [3-((1S,3R)-1-biphenyl-4-ylmethyl-3-ethoxycarbonyl-1-butylcarbamoyl)propionate-(S)-3′-methyl-2′-(pentanoyl{2″-(tetrazol-5-ylate)biphenyl-4′-ylmethyl}amino)butyrate] hemipentahydrate.

29. (canceled)

30. The method according to claim 1, wherein sacubitril and valsartan in a 1:1 molar ratio has been shown to reduce the clinical composite endpoint of death, rehospitalization for HF, LVAD implantation, or listing for cardiac transplant.

31. The method according to claim 1, wherein sacubitril and valsartan in a 1:1 molar ratio is more effective than a medicament comprising a therapeutically effective amount of an ACE inhibitor.

32. The method according to claim 31, wherein sacubitril and valsartan in a 1:1 molar ratio is at least 15% more effective than a medicament comprising a therapeutically effective amount of enalapril in reducing the clinical composite endpoint of death, rehospitalization for HF, LVAD implantation, or listing for cardiac transplant.

33. The method according to claim 32, wherein sacubitril and valsartan in a 1:1 molar ratio has been shown to be statistically superior to a medicament comprising a therapeutically effective amount of enalapril in reducing the clinical composite endpoint of death, rehospitalization for HF, LVAD implantation, or listing for cardiac transplant.
